(19) United States
(12) Patent Application Publication
(10) Pub. No.: US 2024/0257961 A1
(43) Pub. Date: Aug. 1, 2024

(54) METHODS AND SYSTEMS FOR CONTROLLING MEDICAL IMAGING DEVICES

(71) Applicant: WUHAN UNITED IMAGING HEALTHCARE CO., LTD., Wuhan (CN)

(72) Inventors: Jiali YANG, Wuhan (CN); Lu WANG, Shanghai (CN); Zheng DENG, Wuhan (CN)

(73) Assignee: WUHAN UNITED IMAGING HEALTHCARE CO., LTD., Wuhan (CN)

(21) Appl. No.: 18/592,466

(22) Filed: Feb. 29, 2024

Related U.S. Application Data
(63) Continuation of application No. PCT/CN2022/115111, filed on Aug. 26, 2022.

(30) Foreign Application Priority Data
Aug. 30, 2021 (CN) 202111004268.9
Aug. 31, 2021 (CN) 202111011700.7

(51) Int. Cl. G16H 40/60; G06V 40/12; G16H 30/40

(52) U.S. Cl. CPC G16H 40/60; G06V 40/1365; G16H 30/40

(57) ABSTRACT

Embodiments of the present disclosure provide a method and a system for controlling a medical imaging device. The method may include: obtaining second fingerprint information of a user through a collection device; matching the second fingerprint information with first fingerprint information to obtain a matching result; and determining, based on a first mapping relationship and the matching result, an execution function corresponding to the second fingerprint information, and controlling the medical imaging device to perform an operation related to the execution function corresponding to the second fingerprint information.

### CROSS-REFERENCE TO RELATED APPLICATIONS

This application is a Continuation of International Patent Application No. PCT/CN2022/115111, filed on Aug. 26, 2022, which claims priorities to Chinese Patent Application No. 202111011700.7, filed on Aug. 31, 2021, entitled “METHODS AND SYSTEMS FOR CONTROLLING ULTRASOUND DEVICES,” and Chinese Application No. 202111004268.9, filed on Aug. 30, 2021, entitled “SYSTEMS AND METHODS FOR CONTROLLING INTERFACES OF MEDICAL IMAGING DEVICES,” the entire contents of each of which are hereby incorporated by reference.

### TECHNICAL FIELD

The present disclosure relates to the field of medical technology, and in particular to methods and systems for controlling medical imaging devices.

### BACKGROUND

A medical imaging device for diagnosis, such as an ultrasound device used for medical ultrasound detection, provides clinical personnel with images of human tissues. However, an operation panel of the ultrasound device is equipped with multiple buttons corresponding to ultrasound detection functions. During operation, a doctor needs to press multiple buttons on the operation panel corresponding to different functions multiple times and switch between functional interfaces to achieve corresponding ultrasound detection functions. This makes operational control of the ultrasound device cumbersome, leading to lower efficiency in performing relevant operations. Additionally, a user operates the medical imaging device through a user interface where positions and sequences of functional control buttons on a common user interface are fixed. In this setup, there may not be enough space on a main interface when expansion is needed, the functional control buttons may not match a current operation of the user, and the functional control buttons may not be adjusted based on an operation habit of the user.

Therefore, it is desirable to provide a method for controlling a medical imaging device to simplify control operations, enhance a convenience of controlling the medical imaging device, and improve an efficiency of performing relevant operations on the medical imaging device.

### SUMMARY

One embodiment of the present disclosure provides a method for controlling a medical imaging device implemented on a computing device having one or more processors and one or more storage devices. The method may include: obtaining second fingerprint information of a user through a collection device; matching the second fingerprint information with first fingerprint information to obtain a matching result; and determining, based on a first mapping relationship and the matching result, an execution function corresponding to the second fingerprint information, and controlling the medical imaging device to perform an operation related to the execution function corresponding to the second fingerprint information

One embodiment of the present disclosure provides a system for controlling a medical imaging device. The system may include an acquisition module, a matching module, and a determination module. The acquisition module may be configured to obtain second fingerprint information of a user through a collection device. The matching module may be configured to match the second fingerprint information with first fingerprint information to obtain a matching result. The determination module may be configured to determine, based on a first mapping relationship and the matching result, an execution function corresponding to the second fingerprint information, and control the medical imaging device to perform an operation related to the execution function corresponding to the second fingerprint information.

One embodiment of the present disclosure provides an apparatus for controlling a medical imaging device. The apparatus may include at least one storage medium storing one or more computer instructions and at least one processor executing the one or more computer instructions to implement the method for controlling the medical imaging device.

One embodiment of the present disclosure provides a non-transitory computer-readable storage medium. The storage medium stores one or more computer instructions, and when a computer reads the one or more computer instructions, the computer executes the method for controlling the medical imaging device.

One embodiment of the present disclosure provides a system for controlling an interface of a medical imaging device. The system may include a processor and a display device configured to display a user interface including an interface component. The interface component may correspond to one or more functions associated with the medical imaging device or one or more operations performed on the medical imaging device. The processor may be configured to control the medical imaging device based on an operation for the interface component. A type of the interface component includes at least one of an application, an application widget, or a program dock.

One embodiment of the present disclosure provides a method for controlling an interface of a medical imaging device. The method for controlling the interface of the medical imaging device may include displaying a user interface including an interface component through a display device; and receiving an operation for the interface component through one or more processors and controlling the medical imaging device, wherein the interface component may correspond to one or more functions associated with the medical imaging device or one or more operations performed on the medical imaging device and a type of the interface component may include at least one of an application, an application widget, or a program dock.

One embodiment of the present disclosure provides an imaging method. The imaging method may include: displaying a user interface including an interface component through a display device; receiving, through one or more processors, a first operation for the interface component and obtaining information of a scanned object; receiving, through the one or more processors, a second operation for the interface component and obtaining probe information; receiving, through the one or more processors, a third operation for the interface components and obtaining a scanning parameter; and controlling, through the one or more processors, a medical imaging device to perform a scan based on the information of the scanned object, the probe information, and the scanning parameter and obtaining a scanning result.

One embodiment of the present disclosure provides an imaging system. The imaging system may include: a display module configured to display a user interface through a display device, the user interface including an interface component; an object information acquisition module configured to receive, through one or more processors, a first operation for the interface component and obtain information of a scanned object; a probe information acquisition module configured to receive, through the one or more processors, a second operation for the interface component and obtain probe information; a scanning parameter acquisition module configured to receive, through the one or more processors, a third operation for the interface component and obtain a scanning parameter; and a scan result acquisition module configured to control, through the one or more processors, a medical imaging device to perform a scan based on the information of the scanned object, the probe information, and the scanning parameter, to obtain a scanning result.

One embodiment of the present disclosure provides an imaging device. The imaging device may include a display device configured to display a user interface user interface including an interface component, at least one storage medium configured to store one or more computer instructions, and at least one processor configured to execute the one or more computer instructions to implement the imaging method.

One embodiment of the present disclosure provides a non-transitory computer-readable storage medium, wherein the storage medium may store one or more computer instructions, and when a computer reads the one or more computer instructions, the computer may execute the imaging method.

Some embodiments of the present disclosure provide the method and system for controlling the medical imaging device. The second fingerprint information is matched with the first fingerprint information, and upon successful matching, the medical imaging device is controlled via the execution function corresponding to the second fingerprint information. The method eliminates the need for the user to press multiple buttons or use various button combinations to control the medical imaging device, thereby enhancing the convenience and efficiency of controlling the medical imaging device. Additionally, the method reduces the count of buttons on a control panel of the medical imaging device, lowering manufacturing costs and user operational workload.

Some embodiments of the present disclosure provide the system and method for controlling the interface of the medical imaging device. The user can customize a layout of the user interface of the medical imaging device according to needs. By using multiple types of interface components, the user can efficiently and flexibly operate and invoke functions of the medical imaging device. This increases the utilization of interface space, improves interface scalability, reduces unnecessary operations, simplifies operational procedures, facilitates user operations, and enhances the efficiency of tasks related to medical imaging.

### BRIEF DESCRIPTION OF THE DRAWINGS

The present disclosure is further illustrated by way of exemplary embodiments, which are described in detail through the accompanying drawings. These embodiments are not limiting, and in these embodiments, a same numbering indicates a same structure, wherein:

FIG. 1 is a schematic diagram illustrating an exemplary application scenario of a system for controlling a medical imaging device according to some embodiments of the present disclosure;

FIG. 2 is a flowchart illustrating an exemplary process for controlling a medical imaging device according to some embodiments of the present disclosure;

FIG. 3 is a schematic diagram illustrating an exemplary configuration of an execution function of an ultrasound device corresponding to first fingerprint information according to some embodiments of the present disclosure;

FIGS. 4(a) and 4(b) are comparative diagrams illustrating two exemplary operation panels of an ultrasound device according to some embodiments of the present disclosure;

FIG. 5 is a schematic diagram illustrating an exemplary configuration of fingerprint information in an ultrasound device according to some embodiments of the present disclosure;

FIG. 6 is a diagram illustrating an exemplary operation panel of an ultrasound device with two custom buttons according to some embodiments of the present disclosure;

FIG. 7 is a schematic diagram illustrating matching of second fingerprint information with first fingerprint information according to some embodiments of the present disclosure;

FIG. 8 is a schematic diagram illustrating an exemplary state function table according to some embodiments of the present disclosure;

FIGS. 9(a) and 9(b) are schematic diagrams illustrating exemplary control of an ultrasound device through fingerprint information according to some embodiments of the present disclosure;

FIG. 10 is a block diagram illustrating an exemplary system for controlling a medical imaging device according to some embodiments of the present disclosure;

FIG. 11 is a schematic diagram illustrating an exemplary system for controlling an interface of a medical imaging device according to some embodiments of the present disclosure;

FIG. 12 is a flowchart illustrating an exemplary process for configuring a user interface according to some embodiments of the present disclosure;

FIG. 13 is a schematic diagram illustrating exemplary interface components according to some embodiments of the present disclosure;

FIG. 14 is a schematic diagram illustrating exemplary applications according to some embodiments of the present disclosure;

FIG. 15 (a) is a schematic diagram illustrating moving of an exemplary application according to some embodiments of the present disclosure;

FIG. 15 (b) is a schematic diagram illustrating combining of exemplary applications according to some embodiments of the present disclosure;

FIG. 16 is a schematic diagram illustrating exemplary application widgets according to some embodiments of the present disclosure;

FIG. 17 is a schematic diagram illustrating exemplary stacked application widgets according to some embodiments of the present disclosure;

FIG. 18 is a schematic diagram illustrating exemplary interface components according to some embodiments of the present disclosure;

FIG. 19 is a schematic diagram illustrating an exemplary program dock according to some embodiments of the present disclosure;

FIG. 20 is a flowchart illustrating an exemplary process for controlling an interface of a medical imaging device according to some embodiments of the present disclosure; and

FIG. 21 is a flowchart illustrating an exemplary process for imaging according to some embodiments of the present disclosure.

### DETAILED DESCRIPTION

In order to more clearly illustrate the technical solutions of the embodiments of the present disclosure, the accompanying drawings for the description of the embodiments are described below. Obviously, the accompanying drawings in the following description are only some examples or embodiments of the present disclosure, and it is possible for a person of ordinary skill in the art to apply the present disclosure to other similar scenarios in accordance with these accompanying drawings without creative labor. Unless obviously obtained from the context or the context illustrates otherwise, the same numeral in the drawings refers to the same structure or operation.

It should be understood that the terms “system,” “device,” “unit,” and/or “module” are used herein as a way to distinguish between different components, elements, parts, sections, or assemblies at different levels. However, if other words may achieve the same purpose, the terms may be replaced with alternative expressions.

As indicated in the present disclosure and in the claims, unless the context clearly suggests an exception, the words “one,” “a,” “an,” and/or “the” do not refer specifically to the singular but may also include the plural. In general, the terms “include”, “includes”, “including”, “comprise”, “comprising”, and/or “comprise” suggest only the inclusion of clearly identified steps and elements, which do not constitute an exclusive list, and the method or device may also include other steps or elements.

The present disclosure uses flowcharts to illustrate the operations performed by the system according to some embodiments of the present disclosure. It should be understood that the operations described herein are not necessarily executed in a specific order. Instead, they may be executed in reverse order or simultaneously. Additionally, other operations may be added to these processes or certain operations may be removed.

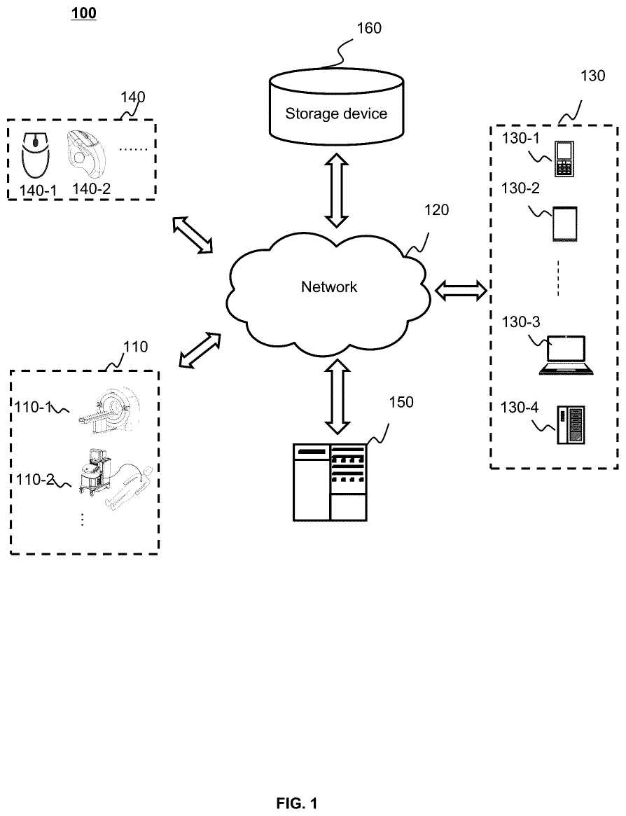

FIG. 1 is a schematic diagram illustrating an exemplary application scenario of a system for controlling a medical imaging device according to some embodiments of the present disclosure.

A system 100 for controlling a medical imaging device may include a medical imaging device 110, a network 120, at least one terminal 130, an input device 140, a processing device 150, and a storage device 160. The components of the system 100 for controlling the medical imaging device (hereinafter referred to as the system 100) may be interconnected via the network 120. For example, the medical imaging device 110 and the at least one terminal 130 may be connected or communicate through the network 120.

The medical imaging device 110 refers to a device in medical practice that reproduces an internal structure of a human body as an image using various media. For example, the medical imaging device 110 may include a medical X-ray machine 110-1 or an ultrasound device 110-2. As another example, the medical imaging device 110 may include a digital imaging device, an X-ray computed tomography device, a magnetic resonance imaging device, a nuclear medicine imaging device, or any combination thereof. The above examples of the medical imaging device 110 is provided for illustrative purposes only and is not intended to be a limitation on the scope of the medical imaging device 110.

In some embodiments, the system 100 may include a collection device (not shown in FIG. 1). In some embodiments, the collection device may be provided on the medical imaging device 110 or may be a separate device. In some embodiments, the collection device refers to a device for collecting a fingerprint, a palm print, a protrusion on a surface of a glove, a pattern on a prosthesis, a brain wave, iris information, or any combination thereof. For example, the collection device may include a semiconductor capacitance-based collection device, an optical collection device, a semiconductor pressure-sensitive collection device, a temperature difference sensing collection device, an ultrasonic recognition collection device, a micro-optical collection device, a radiofrequency collection device, or any combination thereof.

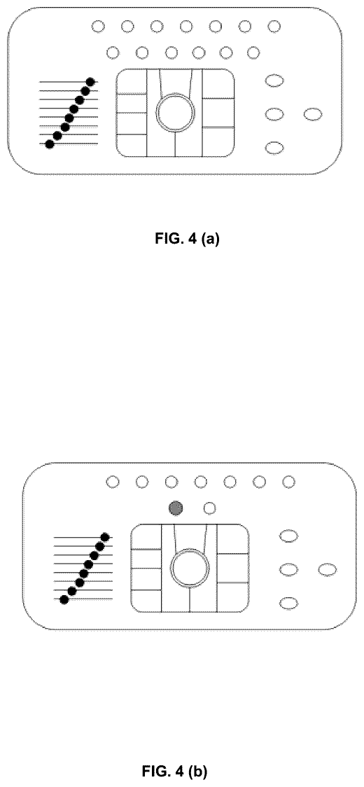

As shown in FIG. 4, taking an ultrasound device as an example, an operation panel of the existing ultrasound device includes multiple buttons (as shown in FIG. 4(a)). During an operation of the ultrasound device, a doctor needs to press multiple buttons on the operation panel corresponding to different functions and switch back and forth between multiple functional interfaces, greatly increasing a workload for the doctor.

To address the above issues, in some embodiments, the collection device may be provided on the medical imaging device 110. For example, inside one or more buttons provided on an ultrasound device (e.g., the gray button as shown in FIG. 4(b), the two gray buttons as shown in FIG. 6), there may be a collection unit for capturing an image and a sensor unit for capturing a pressing parameter. The collection unit and the sensor unit may communicate with the medical imaging device 110 through an interface (e.g., a USB interface). For example, the collection unit may include a fingerprint collection unit, a palm print collection unit, a brain wave collection unit, an iris collection unit, and the sensor unit may include a pressing count collection unit, a pressing time collection unit, a pressing angle collection unit, and any other units for collecting pressing parameters.

In some embodiments, the collection device may also include a camera. When the user wears gloves and contacts the collection device, finger(s) of the user pressed on the medical imaging device and/or the pressing parameter(s) may be recognized based on the camera. In some embodiments, the collection device may also include a touchscreen provided on the medical imaging device 110. For example, relative to a main screen of the medical imaging device 110, the collection device may serve as the secondary screen and be installed side by side with the main screen on the medical imaging device 110.

In some embodiments, a collection device based on a conductive principle may be used to collect the fingerprint and the palm print. In some embodiments, collection devices based on different principles (e.g., ultrasonic collection, optical collection) may be used to collect the fingerprint, the palm print, the protrusion on the surface of the glove, the pattern on the prosthesis, etc.

The collection device may perform collection in various ways. For example, when fingerprints of the user come into contact with the collection device, the collection device (e.g., the semiconductor capacitance-based collection device) may generate different capacitance values based on varying distances between positions of the fingerprints of the user and the collection device, thereby completing collection of the fingerprints. As another example, when the user wearing a glove with a protrusion on a surface of the glove contacts the collection device, the collection device (e.g., the ultrasonic recognition collection device) may emit a signal of a specific frequency to a part of the glove in contact with the collection device, and complete the collection of the protrusion on the surface of the glove based on a reflected signal.

The medical imaging device 110 may be used to perform a scan on an object for diagnostic imaging. The medical imaging device 110 may be used to view images of an internal tissue of the object to assist the doctor in disease diagnosis. The medical imaging device 110 (e.g., the ultrasound device) may send a high-frequency sound wave (e.g., ultrasound) into the object using a probe to generate an ultrasound image. In some embodiments, the object may include a biological object and/or a non-biological object. For example, the object may consist of a specific part of the human body, such as the neck, chest, abdomen, etc., or a combination thereof. As another example, the object may be a patient awaiting scanning by the medical imaging device 110. In some embodiments, a medical image may include an ultrasound image, a digital image, an X-ray computed tomography image, a magnetic resonance image, etc. In some embodiments, the ultrasound image may include at least one of a brightness mode (B-mode) image, a color mode (C-mode) image, a motion mode (M-mode) image, a Doppler mode (D-mode) image, and an elastography mode (E-mode) image. In some embodiments, the medical image may include a two-dimensional (2D) image or a three-dimensional (3D) image.

In some embodiments, the medical image (e.g., the ultrasound image) obtained by the medical imaging device 110 may be sent to the processing device 150 for further analysis. Alternatively or additionally, the medical image obtained by the medical imaging device 110 may be sent to a terminal (e.g., the at least one terminal 130) for display and/or to a storage device (e.g., the storage device 160) for storage.

The network 120 may include any suitable network that facilitates information and/or data exchange within the system 100. In some embodiments, at least one component (e.g., the medical imaging device 110, the processing device 150, the storage device 160, the at least one terminal 130) of the system 100 may exchange information and/or data with at least one other component through the network 120. For example, the processing device 150 may obtain fingerprint information from the medical imaging device 110 through the network 120. The network 120 may be and/or include a public network (e.g., the Internet), a private network (e.g., a local area network (LAN), a wide area network (WAN)), a wired network (e.g., a wireless LAN), an Ethernet, a wireless network (e.g., an 802.11 network, a Wi-Fi network), a cellular network (e.g., a Long-Term Evolution (LTE) network), a frame relay network, a virtual private network (“VPN”), a satellite network, a telephone network, a router, a hub, a switch, a server computer, and/or any combination thereof. For example, the network 120 may include a cable network, a wired network, a fiber optic network, a telecommunication network, an intranet, a wireless LAN (WLAN), a metropolitan area network (MAN), a public switched telephone network (PSTN), a Bluetooth™ network, a ZigBee™ network, a near-field communication (NFC) network, or any combination thereof. In some embodiments, the network 120 may include one or more network access points. For example, the network 120 may include a wired and/or a wireless network access point such as a base station and/or an internet exchange point, where one or more components of the system 100 may connect to the network 120 via the wired and/or the wireless access point to exchange data and/or information.

The at least one terminal 130 may include a mobile device 130-1, a tablet computer 130-2, a laptop computer 130-3, a desktop computer 130-4, etc., or any combination thereof. In some embodiments, the at least one terminal 130 may communicate and/or connect with the medical imaging device 110, the processing device 150, and/or the storage device 160. For example, the user may view the ultrasound image generated by the medical imaging device 110 through the at least one terminal 130.

The input device 140 refers to a device that user may use to input a command. For example, the input device 140 may include a mouse 140-1, a trackball 140-2, a touchpad, a keyboard, a slide stick, etc.

In some embodiments, the command may be input into the processing device 150 through the input device 140, and the processing device 150 may control the medical imaging device 110 via the network 120. The medical imaging device 110 may process accordingly based on the command and display a processing result through the at least one terminal 130. In some embodiments, the terminal 130 and the input device 140 may be integrated as one device, such as a device with a touchscreen, and the command may be input directly through the display device. In some embodiments, the terminal 130, the input device 140, and the processing device 150 may be integrated as one device, such as an all-in-one computer with a touchscreen or a laptop with a touchscreen.

The processing device 150 may process data and/or information obtained from the medical imaging device 110, the at least one terminal 130, and/or the storage device 160. For example, the processing device 150 may obtain fingerprint information of the user from the medical imaging device 110. As another example, the processing device 150 may obtain the fingerprint information of the user from the storage device 160. In some embodiments, the processing device 150 may be a single server or a server group, which may be centralized or distributed. In some embodiments, the processing device 150 may be local or remote. For example, the processing device 150 may access information and/or data from the medical imaging device 110, the storage device 160, and/or the at least one terminal 130 through the network 120. As another example, the processing device 150 may directly connect to the medical imaging device 110, the at least one terminal 130, and/or the storage device 160 to access information and/or data. In some embodiments, the processing device 150 may be implemented on a cloud platform. For example, the cloud platform may include a private cloud, a public cloud, a hybrid cloud, a community cloud, a distributed cloud, an inter-cloud, a multi-cloud, or any combination thereof.

The storage device 160 may store data, instructions, and/or any other information. In some embodiments, the storage device 160 may store data obtained from the medical imaging device 110, the at least one terminal 130, and/or the processing device 150. In some embodiments, the storage device 160 may store data and/or instructions for performing the exemplary methods described in the present disclosure. The storage device 160 may include a high-capacity storage device, a removable storage device, a volatile read-write storage device, a read-only memory (ROM), or any combination thereof. Exemplary high-capacity storage devices may include a disk, a CD, a solid-state drive (SSD), etc. Exemplary removable storage devices may include a flash drive, a floppy disk, a CD, a memory card, a zip disk, a tape, etc. Exemplary volatile read-write storages may include a random access memory (RAM). Exemplary RAMs may include a dynamic random access memory (DRAM), a double data rate synchronous dynamic random access memory (DDR SDRAM), a static random access memory (SRAM), a thyristor random access memory (T-RAM), a zero-capacitor random access memory (Z-RAM), etc. Exemplary ROMs may include a mask ROM (MROM), a programmable ROM (PROM), an erasable programmable ROM (EPROM), an electrically erasable programmable ROM (EEPROM), a compact disc ROM (CD-ROM), a digital versatile disc ROM (DVD-ROM), etc.

In some embodiments, the storage device 160 may be connected to the network 120 to communicate with one or more components (e.g., the processing device 150, the at least one terminal 130) of the system 100. One or more components of the system 100 may access data or instructions stored in the storage device 160 via the network 120. In some instances, the storage device 160 may be directly connected to or communicate with one or more other components (e.g., the processing device 150, the at least one terminal 130) of the system 100. In some embodiments, the storage device 160 may be part of the processing device 150.

It should be noted that the above description is provided for illustrative purposes only and is not intended to limit the scope of the present disclosure. Those skilled in the art may make various changes and modifications based on the guidance provided in this disclosure. The features, structures, methods, and other features of exemplary embodiments described in the present disclosure may be combined in various ways to obtain additional and/or alternative exemplary embodiments. For example, the storage device 160 may include a data storage device that is part of a cloud computing platform. However, these changes and modifications do not depart from the scope of the present disclosure.

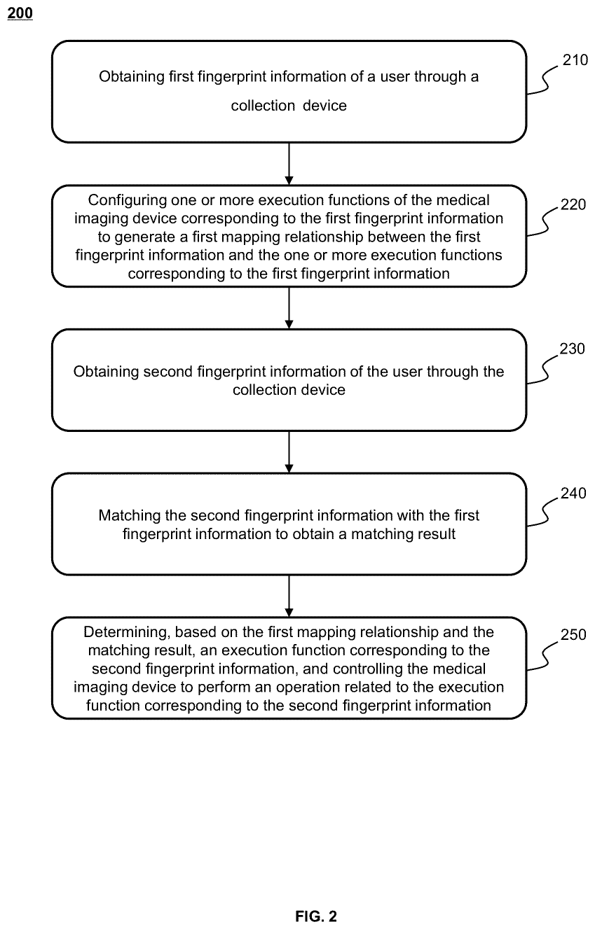

FIG. 2 is a flowchart illustrating an exemplary process for controlling a medical imaging device according to some embodiments of the present disclosure.

As shown in FIG. 2, taking an ultrasound device as an example, process 200 includes the following operations. In some embodiments, process 200 may be executed by the processing device 140. In 210, first fingerprint information of a user may be obtained through a collection device. In some embodiments, operation 210 may be executed by an acquisition module 1010.

Fingerprint information refers to information related to one or more fingerprints of the user, such as a fingerprint image, a fingerprint entry mode, etc. In some embodiments, the fingerprint information may reflect an identity of the user.

In some embodiments, the first fingerprint information may include the fingerprint image and information related to the fingerprint image. In some embodiments, the information related to the fingerprint image may include a target object corresponding to the fingerprint image. For any fingerprint image, the fingerprint image comes from the corresponding target object. In some embodiments, the target object corresponding to the fingerprint image may include a finger, a palm, a glove (e.g., a glove worn by the user), a prosthesis of the user, or any combination thereof. For example, for a user, the fingerprint image may include a finger fingerprint of the user, a palm print of the user, a protrusion on a surface of the glove worn by the user, a pattern on the prosthesis of the user, or any combination thereof.

In some embodiments, the target object corresponding to the fingerprint image is related to an accuracy requirement of fingerprint information recognition. In some embodiments, the accuracy requirement of fingerprint information recognition may be determined based on an operation of the ultrasound device. For example, during user authentication with the ultrasound device (e.g., when the user starts operating the ultrasound device, the system 100 may need to collect the fingerprint information of the user to identify the identity of the user), the accuracy requirement of fingerprint information recognition may be relatively high. In some scenarios, the user (e.g., a doctor) may control the ultrasound device with one hand or wearing a glove. In such cases, a convenience of operating the ultrasound device needs to be considered, and the accuracy requirement of fingerprint information recognition may be relatively low. In some embodiments, the convenience of operating the ultrasound device may be enhanced by lowering the accuracy requirement of recognizing the first fingerprint information. For example, the accuracy requirement of recognizing the first fingerprint information may be lowered to only identify the finger corresponding to the first fingerprint information. That is, a current target object corresponding to the first fingerprint information is a finger of the user, and the ultrasound device may be controlled to execute a corresponding operation by recognizing only the finger of the user. More descriptions regarding controlling the ultrasound device to perform the corresponding operation based on the fingerprint information may be found in operation 250 and the related descriptions thereof.

In some embodiments, the information related to the fingerprint image may include a pressing parameter of the target object. The pressing parameter refers to a parameter generated when the target object presses the collection device. In some embodiments, the pressing parameter may include a count of presses, a pressing strength, a pressing duration, a pressing angle, or any combination thereof.

In some embodiments, the acquisition module 1010 may obtain the first fingerprint information of the user through the collection device. In some embodiments, the acquisition module 1010 may be the collection device itself or a module configured to control the collection device. In some embodiments, the collection device may be a touchscreen provided on the ultrasound device. More descriptions regarding the collection device may be found in FIG. 1 and the related descriptions thereof.

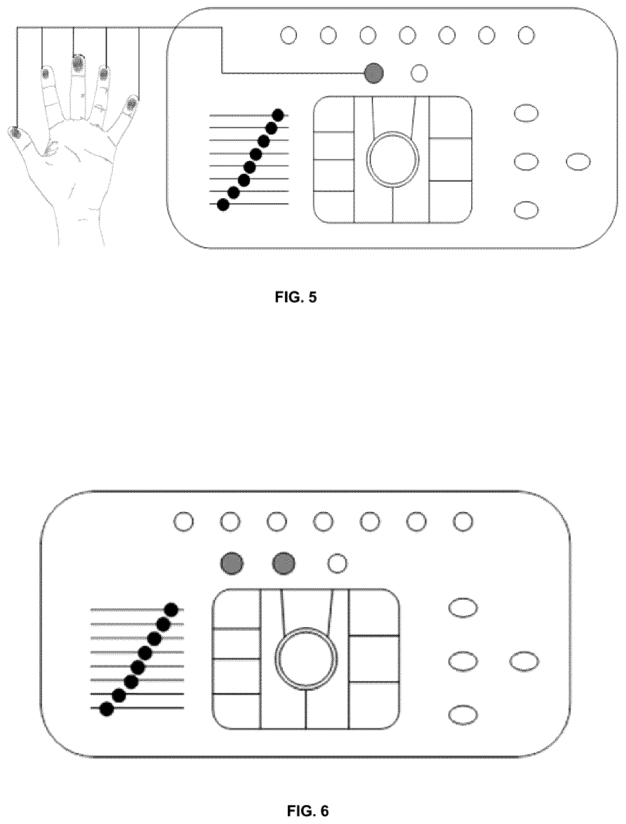

As shown in FIG. 5, the user may press the finger on a capacitive contact end (e.g., a capacitive groove, the capacitive button in FIG. 5) of the collection device, allowing the collection device to capture the fingerprint image of uneven lines on a front skin of the fingertip and obtain corresponding pressing parameters through an internal sensor of the collection device.

The acquisition module 1010 may also obtain the first fingerprint information of the user in other ways. In some embodiments, the acquisition module 1010 may obtain the first fingerprint information from the storage device 150, which stores historical data containing multiple first fingerprint information, via the network 120. There is no limitation on the way of obtaining the first fingerprint information in this embodiment.

By using various types of collection devices to collect first fingerprint information containing diverse information, a comprehensiveness, an accuracy, and a diversity of the first fingerprint information collection can be ensured. This helps avoid situations where the first fingerprint information is incomplete or inaccurate, laying a foundation for subsequent convenient, accurate, and efficient control of the ultrasound device through the fingerprint information of the user.

In 220, one or more execution functions of the ultrasound device corresponding to the first fingerprint information may be configured to generate a first mapping relationship between the first fingerprint information and the one or more execution functions. In some embodiments, operation 220 may be executed by a generation module 1020.

The execution function of the ultrasound device may be any operation functions related to an ultrasound scanning process and/or a scanning result, or all functions that may be controlled by keys on a control panel of the ultrasound device. In addition, the execution function may be one or more commonly used execution functions selected by the doctor from the above execution functions.

In some embodiments, the execution function of the ultrasound device may include ultrasound image optimization, ultrasound measurement, interface switching, ultrasound image annotation, ultrasound image display mode, ultrasound image switching, coded harmonic ultrasound imaging, scanning guidance page displaying, ultrasound probe position adjusting, voice assistant invoking, ultrasound probe switching, ultrasound clinical application, patient registration and management, ultrasound reporting, ultrasound image reviewing, or any combination thereof. For example, the ultrasound image optimization refers to the use of the ultrasound device to perform gain processing on an ultrasound image to enhance a display effect of the ultrasound image; the ultrasound measurement refers to the ultrasound device detecting a target region using ultrasound waves; the interface switching refers to switching between various display interfaces or functional interfaces of the ultrasound device; the ultrasound image annotation refers to annotating and marking the ultrasound image; the ultrasound image display modes refer to selecting, switching, and editing display modes of the ultrasound image; the ultrasound image switching refers to switching between generated ultrasound images; the coded harmonic ultrasound imaging refers to imaging a target object using coded harmonic ultrasound by the ultrasound device; the scanning guidance page displaying refers to displaying information related to scan guidance for the user on a screen of the ultrasound device; the ultrasound probe position adjusting refers to changing the position of the ultrasound probe in the target region using the ultrasound device; the voice assistant invoking refers to calling a voice assistant of the ultrasound device to assist the user in operations, for example, the user may perform operations such as the ultrasound image annotation through voice during an ultrasound measurement process; the ultrasound probe switching refers to switching an ultrasound probe used for scanning using the ultrasound device; the ultrasound clinical application refers to using the ultrasound device to make clinical diagnoses for a patient, such as strain analysis for the heart or automatic measurements for obstetrics; the patient registration and management refers to registering and managing patient information, cases, and prescriptions using the ultrasound device; the ultrasound reporting refers to generating and/or viewing reports of an ultrasound scan result using the ultrasound device; the ultrasound image reviewing refers to reviewing a historical ultrasound image using the ultrasound device.

By establishing the first mapping relationship between of the fingerprint information of the user and the one or more execution functions of the ultrasound device, the user can subsequently control the ultrasound device through the fingerprint information. This greatly simplifies user control operations on the ultrasound device, alleviating the original pressure on the user who had to perform manual measurements, annotations, and other workflow tasks simultaneously with controlling the ultrasound device, and effectively optimizing an overall process of controlling the ultrasound device for work execution.

The first mapping relationship may represent a corresponding relationship between the first fingerprint information and corresponding execution function(s) of the ultrasound device. After the user is verified, based on the first mapping relationship, the system 100 may automatically control the ultrasound device to execute the corresponding function(s) associated with the fingerprint information.

In some embodiments, the first fingerprint information may include fingerprint information of multiple fingers of the user. For example, the first fingerprint information may include ten fingerprint images corresponding to the ten fingers of the user.

In some embodiments, the first mapping relationship may include a corresponding relationship between a combination (and/or sequence) of fingerprint information of two or more fingers of the user and corresponding execution function(s). For example, the first mapping relationship may include a corresponding relationship between a combination of fingerprint images of two fingers (e.g., an index finger and a middle finger) of the user and the interface switching function of the ultrasound device. Thus, when the system 100 recognizes the combination of the fingerprint images of the two fingers (e.g., two fingers simultaneously or sequentially pressing the collection device), the system 100 may automatically control the ultrasound device to execute the interface switching function.

In some embodiments, an operation related to the system 100 in recognizing the combination of the fingerprint images of the two fingers of the user may be implemented through the operation panel of the ultrasound device, as shown in FIG. 6. The user may simultaneously or sequentially press two gray buttons for the system 100 to perform the recognition operation described above. The two gray buttons in FIG. 6 represent the aforementioned collection device, and the collection device may include one or more buttons. This configuration allows the user to press multiple buttons of the collection device during the above recognition operation to complete fingerprint recognition. Moreover, through the multiple buttons, it is possible to combine multiple fingerprint information to expand a count of mappings of the execution functions of the ultrasound device. For example, the fingerprint information (e.g., the fingerprint images) of the two fingers may be mapped to the function of displaying different interfaces of the ultrasound device and, based on this mapping, the fingerprint information (e.g., the fingerprint images) of the two fingers may be mapped to the interface switching function of the ultrasound device.

In some embodiments, different combinations of fingerprint images may correspond to different execution functions. For example, fingerprint images of an index finger and a middle finger correspond to the execution function of ultrasound image optimization, and fingerprint images of a middle finger and a ring finger correspond to the execution function of ultrasound measurement. In some embodiments, different arrangements of the same two or more fingerprint images may correspond to different execution functions. For example, for an index finger and a middle finger of the same user, pressing the index finger first and then pressing the middle finger corresponds to the execution function of coded harmonic ultrasound imaging, and pressing the middle finger first and then pressing the index finger corresponds to the execution function of ultrasound measurement.

In some embodiments, the first mapping relationship may include a corresponding relationship between a combination of a fingerprint image of one finger of the user with a pressing parameter and a corresponding execution function. For example, the first mapping relationship may include a corresponding relationship between a combination of a fingerprint image of an index finger the user with a pressing strength of 100N and the ultrasound image optimization function of the ultrasound device. Thus, when the system 100 recognizes the fingerprint image of the index finger and identifies the pressing strength of the index finger as 100N, the system 100 may automatically control the ultrasound device to execute the ultrasound image optimization function. In some embodiments, combinations of a same fingerprint image with different pressing parameters (e.g., one or more of different counts of presses, different pressing strengths, different pressing durations, different pressing angles) may correspond to different execution functions. For example, an index finger of the user pressing continuously twice corresponds to the execution function of ultrasound image optimization, and the index finger of the user pressing continuously three times corresponds to the execution function of ultrasound measurement. More descriptions regarding the pressing parameter may be found in operation 210 and the related descriptions thereof.

In some embodiments, the first mapping relationship may also include a corresponding relationship between the first fingerprint information and multiple execution functions. For example, a specific fingerprint image and/or pressing parameter of the user may simultaneously correspond to the functions of coded harmonic ultrasound imaging and ultrasound image annotation. In some embodiments, the first mapping relationship may also include an execution sequence of multiple execution functions, which may be automatically arranged based on the rationality of the multiple execution functions or set according to user preferences. For example, the specific fingerprint image and/or pressing parameter of the user may simultaneously correspond to coded harmonic ultrasound imaging and ultrasound image annotation, and the execution sequence may be set to first execute the coded harmonic ultrasound imaging function and then the ultrasound image annotation function.

In some embodiments, the system 100 may guide the user to press a target object (e.g., finger, palm, glove, prosthesis, etc.) to a designated position. For example, the system 100 may use voice prompts to guide the user to press the target object into a capacitive groove of the collection device. In some embodiments, after the user presses the target object to the designated position, and the collection device collects the first fingerprint information, the generation module 1020 may configure the execution function(s) of the ultrasound device corresponding to the first fingerprint information. After the user confirms the execution function(s) corresponding to the first fingerprint information, the generation module 1020 may encode and write the execution function(s) into the first fingerprint information to establish the first mapping relationship between the first fingerprint information and the corresponding execution function(s).

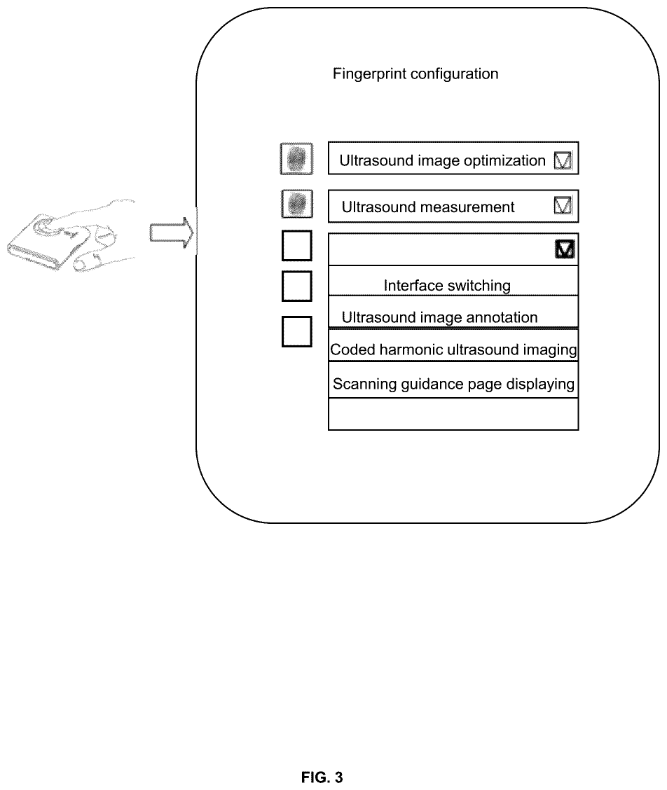

In some embodiments, during a configuration process, a configuration interface of the ultrasound device may display to the user execution functions already configured for different fingerprint information for confirmation or modification. As shown in FIG. 3, a mapping is formed between a first fingerprint image of the user and the ultrasound image optimization function, and another mapping is formed between a second fingerprint image of the user and the ultrasound measurement function. In some embodiments, the users may modify the already configured execution functions.

In some embodiments, during the configuration process, the configuration interface of the ultrasound device may display to the user execution functions that may be configured for different fingerprint information for selection. As shown in FIG. 3, when the user configures execution function(s) corresponding to a third fingerprint image, the collection device may transmit the third fingerprint image of the user to the ultrasound device. Subsequently, the configuration interface of the ultrasound device may display checkboxes for multiple execution functions in the corresponding function selection column, such as interface switching, ultrasound image annotation, coded harmonic ultrasound imaging, scanning guidance page displaying, etc. The user may make selections on the configuration interface to confirm the execution function(s) corresponding to the third fingerprint image. For example, selecting the interface switching function indicates that the user confirms a mapping between the third fingerprint image and the interface switching function. It is important to note that FIG. 3 only shows a simple example of configuring the execution function corresponding to the first fingerprint information and does not limit the scope of the present disclosure to the example given.

In some embodiments, the user may use two or more fingerprint images, pressing parameters, or combinations thereof to configure the corresponding relationship, similar to the configuring process using a single fingerprint image. For example, the user may simultaneously press a designated position with two fingers, then the generation module 1020 may initiate the configuration process and display available functions on the configuration interface of the ultrasound device to the user. Once the user makes selections and confirms, the generation module 1020 may establish a mapping between the fingerprint images obtained from the simultaneous use of the two fingers and the corresponding executed function(s).

In some embodiments, the generation module 1020 may provide the user with a functional selection interface to determine whether to associate different orders of fingerprint image combinations, allowing the user to make corresponding configurations based on needs. If the user chooses to associate different orders of fingerprint image combinations, then, when fingerprint images are selected, different orders in which the fingerprint images appear correspond to different executed functions. If the user chooses not to associate different orders of fingerprint image combinations, then, when fingerprint images are selected, different orders in which the fingerprint images appear correspond to a same executed function. In other words, the order of appearance of the fingerprint images is not considered a determining factor for the executed function.

In some embodiments, the generation module 1020 may display to the user pressing parameter information of the fingerprint image collected by the collection device. The user may consider various factors such as the fingerprint image, the pressing parameter, etc., to configure the corresponding execution function(s). In some embodiments, the user may adjust the pressing parameter.

In some embodiments, after the user complete the configuration process, the generation module 1020 may store the fingerprint information of the user and the corresponding execution functions (i.e., the first mapping relationship). For example, the generation module 1020 may package the fingerprint information of the user into personal fingerprint information of the user and store the personal fingerprint information in a personal fingerprint database of the user. In some embodiments, after the generation module 1020 completes the packaging and storage of the fingerprint information of the user, the system 100, when recognizing the fingerprint information of the user, may match the fingerprint information with the personal fingerprint information of the user in the personal fingerprint database.

Through the above process, forming the first mapping relationship by associating the first fingerprint information in various combinations with at least one execution function allows the first fingerprint information to correspond to multiple execution functions or various combinations of execution functions, laying a foundation for quickly and accurately controlling the ultrasound device subsequently using fingerprint information. Additionally, packaging and storing the fingerprint information of the user in the personal fingerprint database reduces the computational load of the system 100 and improves operational efficiency when identifying the fingerprint information of the user in the future.

In 230, second fingerprint information of the user may be obtained through the collection device. In some implementations, operation 230 may be performed by a second acquisition module 1030.

In some embodiments, the second fingerprint information may be a part of the first fingerprint information. In some embodiments, the second fingerprint information may be different from the first fingerprint information. For example, the second fingerprint information and the first fingerprint information may correspond to different fingers of a same user, or correspond to different users.

The process of obtaining the second fingerprint information may be similar to the obtaining of the first fingerprint information. More descriptions regarding the obtaining of the second fingerprint information may be found in 210 and the related descriptions thereof.

In 240, the second fingerprint information may be matched with the first fingerprint information to obtain a matching result. In some embodiments, operation 240 may be performed by a matching module 1040.

In some embodiments, the matching module 1040 may match the second fingerprint information with the first fingerprint information to obtain the matching result. In some embodiments, the matching module 1040 may determine the matching result by comparing whether the second fingerprint information shares at least some common features with the first fingerprint information. For example, if the second fingerprint information only includes a fingerprint image and the fingerprint image is similar to the fingerprint image in the first fingerprint information, a successful match is indicated, and the matching result is “yes”. As another example, if the second fingerprint information includes a fingerprint image and a pressing parameter, and the first fingerprint information includes a similar fingerprint image and an identical or similar pressing parameter, a successful match is indicated and the matching result is “yes”. More descriptions regarding the matching result may be found in FIG. 4 and the related descriptions thereof.

In 250, the execution function corresponding to the second fingerprint information may be determined based on the first mapping relationship and the matching result, and the ultrasound device may be controlled to perform an operation related to the execution function corresponding to the second fingerprint information. In some embodiments, operation 250 may be performed by a determination module 1050.

In some embodiments, if the matching result is “yes”, it indicates that the first fingerprint information includes target fingerprint information that matches (e.g., is the same, similar, or identical) the second fingerprint information. The determination module 1050 may designate the execution function corresponding to the target fingerprint information as the execution function corresponding to the second fingerprint information and control the ultrasound device to perform the operation related to the execution function corresponding to the second fingerprint information.

In some embodiments, if the matching result is “no”, it indicates that there is no target fingerprint information in the first fingerprint information that matches (e.g., is the same, similar, or identical to) the second fingerprint information. The determination module 1050 may re-determine the execution function corresponding to the second fingerprint information in other ways. More descriptions regarding the matching result and the determination of the execution function corresponding to the second fingerprint information may be found in FIG. 4 and the related descriptions thereof.

In some embodiments, the operation related to the execution function corresponding to the second fingerprint information may include an operation of the execution function itself. For example, if the execution function corresponding to the second fingerprint information is to display a scanning guidance page, the determination module 1050 may control the ultrasound device to display the scanning guidance page on a main screen for the user to view. In some embodiments, the operation related to the execution function corresponding to the second fingerprint information may include an operation related to implementing the execution function. For example, if the execution function corresponding to the second fingerprint information is to display a scanning guidance page, the determination module 1050 may first check whether the ultrasound device has a stored scanning guidance page. If a check result indicates that there is no stored scanning guidance page, the determination module 1050 may control the ultrasound device to prompt the user to record a video or an audio of a scan guidance on the main screen and store the recorded video or audio on a server of the ultrasound device as the content of the scanning guidance page.

In some embodiments, the determination module 1050 may control the ultrasound device to perform corresponding operations through means other than fingerprint information. For example, in some embodiments, the determination module 1050 may control the ultrasound device based on electroencephalogram (EEG) information. Different EEG information of the user may correspond to different execution functions of the ultrasound device. For example, the EEG information may indicate a highly active state or a generally active state. Merely by way of example, if the EEG information indicates a highly active state, the executed function corresponding to the ultrasound device may be ultrasound measurement. When the determination module 1050 receives EEG information indicating a highly active state, the determination module 1050 may control the ultrasound device to perform an operation corresponding to ultrasound measurement. In some embodiments, the determination module 1050 may control the ultrasound device based on iris information. Different iris information of the user may correspond to different execution functions of the ultrasound device. For example, the iris information may represent position(s) of one or more points. Merely by way of example, if the iris information corresponds to positions of two points, the corresponding function may be ultrasound image optimization. When the system 100 obtains the iris information indicating the positions of the two points, the determination module 1050 may control the ultrasound device to perform an operation corresponding to ultrasound image optimization.

Through the above manners, the second fingerprint information may be comprehensively and accurately matched with the first fingerprint information. After a successful match, the ultrasound device may be controlled through the execution function corresponding to the second fingerprint information, eliminating the need to press multiple buttons or use various button combinations to control the ultrasound device. This enhances the convenience and efficiency of controlling the ultrasound device, reduces a count of buttons on the control panel of the ultrasound device (as shown in FIG. 4), lowers manufacturing costs, and reduces the workload for user operations. In case of an unsuccessful match, the system 100 may flexibly determine new execution functions for the second fingerprint information to control the ultrasound device, achieving the aforementioned benefits. Additionally, because fingerprint recognition is more sensitive and faster than buttons, accurate and efficient fingerprint information collection and control of the ultrasound device can be achieved without adding additional attachments to the control panel of the ultrasound device, apart from the collection device.

In some embodiments, the determination module 1050 may control the at least one terminal 130 to display a user interface that includes an interface component corresponding to the second fingerprint information. The interface component may be configured to correspond to a function of the medical imaging device and/or an operation performed on the medical imaging device, and a type of the interface component includes at least one of an application, an application widget, or a program dock.

In some embodiments, if the first fingerprint information includes the target fingerprint information that matches (e.g., is the same, similar, or identical to) the second fingerprint information, the determination module 1050 may designate an interface component corresponding to the target fingerprint information as the interface component corresponding to the second fingerprint information and display a user interface that includes the interface component.

In some embodiments, if there is no target fingerprint information in the first fingerprint information that matches (e.g., is the same, similar, or identical to) the second fingerprint information, the determination module 1050 may re-determine the interface component corresponding to the second fingerprint information through other means. For example, the determination module 1050 may determine the interface component corresponding to the second fingerprint information based on factory settings or default settings.

It should be noted that the description of the process 200 above is only for illustration and explanation, and does not limit the scope of the present disclosure. For those skilled in the art, various modifications and changes may be made to the process 200 under the guidance of the present disclosure. However, these modifications and changes are still within the scope of the present disclosure. In some embodiments, the accuracy requirement for the first fingerprint information collected in operation 210 and/or the second fingerprint information collected in operation 230 may be relatively low. The user may not need to pay too much attention to the collection process (e.g., the user does not need to place finger(s) strictly on the collection device to obtain clear fingerprint image information; instead, the user may casually place the finger(s) on the collection device). In some embodiments, the collection device may be provided with a camera to assist in analyzing the collected fingerprint information and identifying the corresponding target object, thereby allowing the system 100 to quickly determine the corresponding execution function based on an existing mapping relationship (e.g., the first mapping relationship). This approach avoids the user investing too much effort in the fingerprint information collection process, allowing the user to focus more on the execution function of the ultrasound device and patient scanning, reducing the burden of the user, simplifying operations, and improving a control efficiency of the ultrasound device.

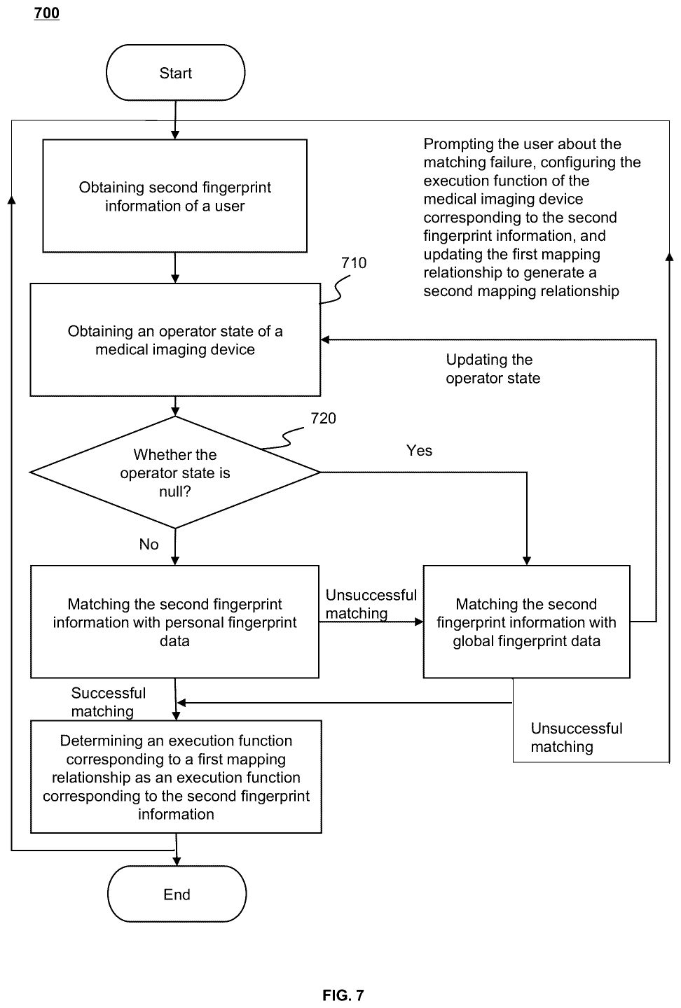

FIG. 7 is a schematic diagram illustrating matching of second fingerprint information with first fingerprint information according to some embodiments of the present disclosure. As shown in FIG. 7, process 700 may include the following operations. In some embodiments, the process 700 may be executed by the processing device 140.

In 710, an operator state of a medical imaging device (e.g., an ultrasound device) may be obtained. In some embodiments, operation 710 may be performed by the determination module 1050.

The operator state refers to a state whether the ultrasound device is currently being used by a user. A null operator state indicates that the ultrasound device is not being used by any user, while a non-null operator state indicates that the ultrasound device is currently being used by a user. In some embodiments, the user corresponding to the operator state may be a default user of the ultrasound device.

In some embodiments, when the second fingerprint information is matched with the first fingerprint information, the determination module 1050 may obtain the operator state of the ultrasound device. In some embodiments, the determination module 1050 may retrieve, through the network 120 or an interface, information about whether the ultrasound device is currently being used (e.g., whether there is a logged-in user using the ultrasound device) from a server or a database of the ultrasound device, thereby determining the operator state of the ultrasound device. For example, the determination module 1050 may retrieve state data (e.g., an operator, a start and/or an end usage time, an operation performed during usage, etc.) of the ultrasound device from the server of the ultrasound device and determine whether the operator state of the ultrasound device is null based on the state data. In some embodiments, the determination module 1050 may directly read the operator state from the ultrasound device.

In 720, in response to determining that the operator state is not null, the second fingerprint information may be matched with personal fingerprint data corresponding to the operator state to obtain a first matching result, and in response to determining that the operator state is null, the second fingerprint information may be matched with global fingerprint data to obtain a second matching result. In some embodiments, operation 720 may be performed by the determination module 1050.

In some embodiments, when the operator state is not null, the determination module 1050 may match the second fingerprint information with the personal fingerprint data corresponding to the user to obtain the first matching result. In some embodiments, the user corresponding to the operator state may be the default user for the ultrasound device. Therefore, when the operator state is not null, the determination module 1050 may invoke the personal fingerprint database of the default user and match the second fingerprint information with the personal fingerprint data in the personal fingerprint database, thereby prioritizing verifying whether the second fingerprint information matches fingerprint information (e.g., the first fingerprint information) of the default user. If the second fingerprint information matches the fingerprint information, the first matching result is successful; if the second fingerprint information does not match the fingerprint information, the first matching result is unsuccessful.

In some embodiments, when the first matching result is successful, the determination module 1050 may determine first target fingerprint information in the personal fingerprint data (e.g., the first fingerprint information) that matches (e.g., is the same, similar, or close to) the second fingerprint information. Based on a mapping relationship of the personal fingerprint data (e.g., a first mapping relationship), the determination module 1050 may determine a first target execution function corresponding to the first target fingerprint information and designate the first target execution function as the execution function corresponding to the second fingerprint information. A successful first matching result indicates that the second fingerprint information matches (e.g., is the same, similar, or close to) the personal fingerprint data (e.g., of the default user). In other words, the user corresponding to the second fingerprint information is the operator corresponding to the operator state, and accordingly, the user may execute the execution function corresponding to the first mapping relationship to control the ultrasound device to perform the corresponding operation.

As mentioned earlier, in some scenarios, the convenience of controlling the ultrasound device can be enhanced by reducing the accuracy requirement for fingerprint information recognition. In some embodiments, the determination module 1050 may obtain an operational status of the ultrasound device during any time periods to determine whether to lower the accuracy requirement for fingerprint information recognition. For example, if the determination module 1050 observes that all users who have used the ultrasound device in the past forty-eight hours are User A, the determination module 1050 may determine that User A is likely to be the next user to use the ultrasound device. The determination module 1050 may then decide to lower the accuracy requirement for fingerprint information recognition for User A. For example, when collecting or recognizing the fingerprint information of User A, the determination module 1050 may only recognize a finger, a glove, a palm print, etc., of User A as an alternative. More descriptions regarding lowering the accuracy requirement for fingerprint information recognition may be found in operation 210 and the relevant descriptions thereof.

In some embodiments, when the first matching result is unsuccessful, the determination module 1050 may match the second fingerprint information with the global fingerprint data to obtain the second matching result. In some embodiments, the global fingerprint data may include fingerprint information data corresponding to all users who have configured fingerprint information in a current ultrasound device. In other words, a user corresponding to the global fingerprint data is a user who has configured fingerprint information corresponding to the execution function on the current ultrasound device. In some embodiments, a type of the fingerprint information included in the global fingerprint data may be the same as or partially the same as a type of the fingerprint information included in the personal fingerprint data. For example, the global fingerprint data may include a fingerprint image, a pressing parameter, etc.

In some embodiments, the global fingerprint data may also be stored in the storage device 150 and include fingerprint information data corresponding to all the users who have configured fingerprint information in all ultrasound devices. In other words, users corresponding to the global fingerprint data include users who have configured fingerprint information corresponding to the execution function on the current ultrasound device. In this way, for a user who has configured fingerprint information on one ultrasound device, when using another ultrasound device, the fingerprint information on the two ultrasound devices may be matched through the global fingerprint data to control the another ultrasound device using the fingerprint information, eliminating the need for the user to repeatedly configuring fingerprint mapping relationships and increasing a flexibility of use.

Through the above approach, flexibility and comprehensiveness in matching the global fingerprint data with fingerprint information can be increased, thereby enhancing the flexibility and efficiency of controlling the ultrasound device through the fingerprint information.

An unsuccessful first matching result indicates that the second fingerprint information does not match (e.g., is not the same, not similar, or not close) with the first fingerprint information. In other words, the user corresponding to the second fingerprint information is not the user corresponding to the operator state. Accordingly, the determination module 1050 may match the second fingerprint information with the global fingerprint data to determine whether there is fingerprint information in the global fingerprint data that matches (e.g., is the same, similar, or close to) the second fingerprint information. More descriptions regarding the second matching result may be found in the subsequent related descriptions.

In some embodiments, when the operator state is null, the determination module 1050 may match the second fingerprint information with the global fingerprint data to obtain the second matching result. When the operator state is null, the determination module 1050 may invoke a global fingerprint database, match the second fingerprint information with fingerprint information of multiple users in the global fingerprint database to determine whether there is fingerprint information in the global fingerprint data that matches (e.g., is the same, similar, or close to) the second fingerprint information. If the global fingerprint data include fingerprint information that matches the second fingerprint information, the second matching result is successful; if the global fingerprint data does not include fingerprint information that matches the second fingerprint information, the second matching result is unsuccessful.

In some embodiments, when the second matching result is successful, the determination module 1050 may determine second target fingerprint information in the global fingerprint data that matches the second fingerprint information. Based on a mapping relationship (e.g., a mapping relationship between the fingerprint information of the user corresponding to the second target fingerprint information and the execution function) associated with the second target fingerprint information, the determination module 1050 may determine the second target execution function corresponding to the second target fingerprint information and designate the second target execution function as the execution function corresponding to the second fingerprint information. In some embodiments, the determination module 1050 may update the operator state to be not null. In some embodiments, a successful second matching result may indicate that the user corresponding to the second fingerprint information is one of the users in the global fingerprint database (i.e., the user has an existing mapping relationship), and the determination module 1050 may determine the corresponding execution function for the second fingerprint information based on the existing mapping relationship. In some embodiments, the determination module 1050 may also update the operator state to be not null (or non-null).

In some embodiments, when the second matching result is unsuccessful, the determination module 1050 may prompt the user about the matching failure and configure the execution function of the ultrasound device corresponding to the second fingerprint information. The determination module 1050 may update the first mapping relationship to generate a second mapping relationship. An unsuccessful second matching result indicates that there is no fingerprint information in the global fingerprint database that matches (e.g., is the same, similar, or close to) the second fingerprint information. In some embodiments, the user corresponding to the second fingerprint information may not be one of the users in the global fingerprint database, i.e., the user does not have an existing mapping relationship. In some embodiments, the user corresponding to the second fingerprint information may be one of the users in the global fingerprint database, but the mapping relationship of the user is not stored on the server of the ultrasound device, or the fingerprint information of the user is incomplete, resulting in an incomplete mapping relationship for the user and making the second fingerprint information unable to match successfully. For example, the second fingerprint information corresponds to a fingerprint image of a middle finger of the user, while the global fingerprint database only stores a fingerprint image of an index finger of the user. After determining that the second matching result is unsuccessful, the determination module 1050 may configure the execution function of the ultrasound device corresponding to the second fingerprint information through the above-described operation 220, achieving an update to the mapping relationship for the user (e.g., adding the second fingerprint information and corresponding execution function to the first mapping relationship of the user and updating the first mapping relationship to be the second mapping relationship).

Through the above approach, the operator state of the ultrasound device is first obtained. If the operator state is not null, the second fingerprint information is first matched with the personal fingerprint data, reducing a computational load during the fingerprint information matching process and improving a computational speed of the system 100.

It should be noted that the description of process 700 above is only for illustration and explanation and does not limit the scope of the present disclosure. For those skilled in the art, various modifications and changes may be made to process 700 under the guidance of the present disclosure. However, these modifications and changes are still within the scope of the present disclosure.

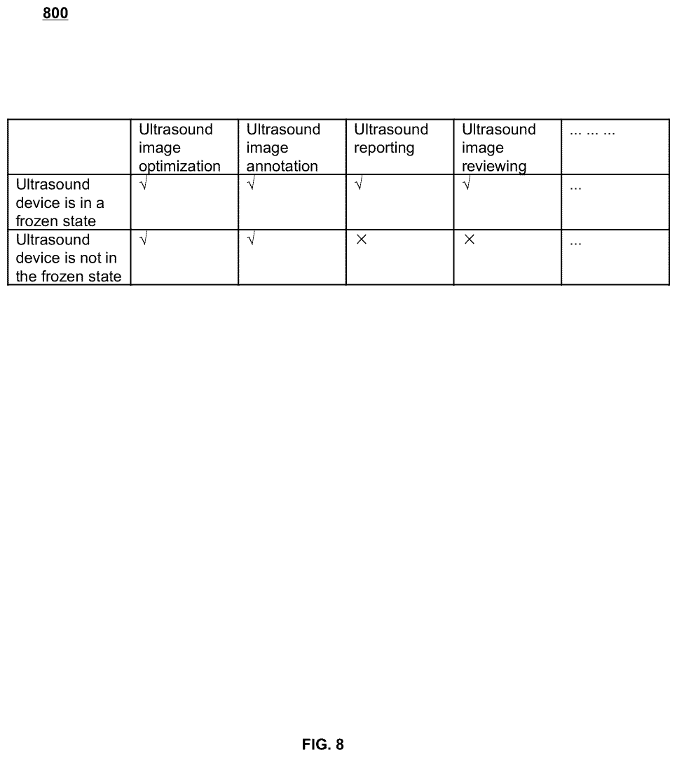

FIG. 8 is a schematic diagram illustrating an exemplary state function table according to some embodiments of the present disclosure.

A mutual exclusion anti-interruption mechanism is a program mechanism set in an ultrasound device. The mutual exclusion anti-interruption mechanism refers to a mechanism where the ultrasound device, when performing operations corresponding to its execution function, may have an ongoing operation that may not be interrupted, or multiple operations may not be performed simultaneously. For example, when the ultrasound device is executing a function of ultrasound image optimization, to achieve the best optimization effect for an ultrasound image, it is necessary to ensure that the ultrasound image optimization function is interrupted as little as possible, which may be achieved by introducing the mutual exclusion anti-interruption mechanism into the ultrasound device. As another example, when the ultrasound device is performing the function of ultrasound image optimization, and the ultrasound image has already been generated, so the ultrasound device may not be able to simultaneously execute related operations for a function of scanning guidance page displaying, which may be achieved by introducing the mutual exclusion anti-interruption mechanism into a control flow of the ultrasound device.

In some embodiments, the determination module 1050 may determine, based on the mutual exclusion anti-interruption mechanism, whether to execute a related function and/or operation. For example, when controlling the ultrasound device to perform the ultrasound image optimization function, the determination module 1050 may determine, based on the mutual exclusion anti-interruption mechanism, whether to control the ultrasound device to simultaneously execute functions such as interface switching and ultrasound measurement, thereby ensuring an optimal control effect and an optimal execution effect on the ultrasound device and ensuring safety of an ultrasound operation and normal execution of an ultrasound function.

In some embodiments, the determination module 1050 may determine whether to execute an operation related to the execution function corresponding to the second fingerprint information based on various mutual exclusion anti-interruption mechanisms. For example, the determination module 1050 may determine, based on multiple time intervals between execution functions, whether to perform the operation related to the execution function corresponding to the second fingerprint information. As another example, the determination module 1050 may determine, based on a recent operating condition of a user on the ultrasound device, whether to control the ultrasound device to perform the operation related to the execution function corresponding to the second fingerprint information.

In some embodiments, the determination module 1050 may use a state function table 800 to determine whether to execute the operation related to the execution function corresponding to the second fingerprint information.

The state function table 800 may be stored internally in the ultrasound device and used to determine whether multiple execution functions of the ultrasound device may be executed simultaneously. The state function table 800 may take various forms and/or contents. Merely by way of example, as shown in FIG. 8, the state function table 800 may include conditions such as: when the ultrasound device is in a frozen state, ultrasound image optimization, ultrasound image annotation, ultrasound reporting, and/or ultrasound image reviewing may be executed; when the ultrasound device is not in the frozen state, ultrasound image optimization, ultrasound image annotation may be executed, but ultrasound reporting, ultrasound image review, etc., may not be executed. In the present disclosure, the ultrasound device being in the frozen state means that one or more components and/or programs of the ultrasound device are frozen and may not be operated within a specified time. In some embodiments, the state function table may be preset and adjusted and/or updated based on usage requirements or actual usage conditions of the ultrasound device. In some embodiments, the state function table may be stored in the storage device 150.

In some embodiments, the determination module 1050 may determine whether the ultrasound device is in the frozen state to obtain a first judgment result and, based on the first judgment result, obtain a second judgment result. The first judgment result may indicate whether the ultrasound device is in the frozen state. The second judgment result may indicate whether to perform the operation related to the execution function corresponding to the second fingerprint information. For example, if it is determined that the ultrasound device is in the frozen state, the first judgment result is that the ultrasound device is in the frozen state, and based on the first judgment result, the second judgment result is that ultrasound image optimization, ultrasound image annotation, ultrasound reporting, and ultrasound image reviewing functions may be executed.

Through the above method, setting up a state function table in the ultrasound device can prevent interruptions during the execution of important functions and also prevent the execution of some mutually exclusive operations. For example, the ultrasound image optimization function and the scanning guidance page displaying function may be mutually exclusive and may not be executed simultaneously. The state function table provides a basis for determining whether to execute the operation related to the execution function corresponding to the second fingerprint information, while reducing an impact of mis-touch and mis-operation.

In some embodiments, the system 100 may control the ultrasound device to execute different functions and/or display different interfaces based on different pressing parameters of one or more fingers of the user. In some embodiments, a mode of the display may be one or more of screen display, voice broadcast, or a combination thereof.

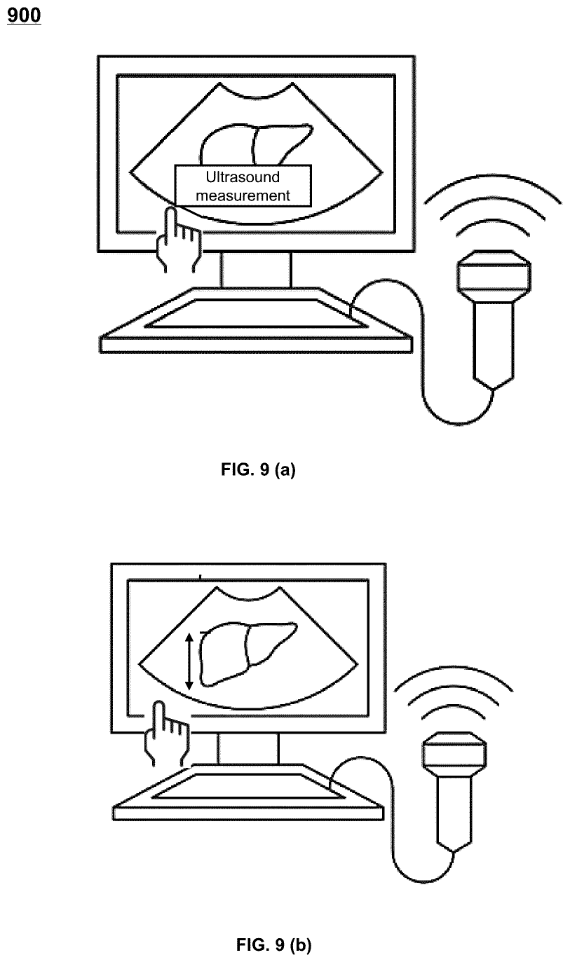

In some embodiments, different functions may be executed and/or different interfaces may be displayed based on magnitudes of the pressing parameters of the user's finger(s). In some embodiments, when at least one pressing parameter of the user (e.g., one finger) on a collection device is less than a threshold, the system 100 may control the ultrasound device to display the execution function corresponding to the fingerprint information (e.g., the second fingerprint information) to the user. In some embodiments, when at least one pressing parameter of the user (e.g., one finger) on the collection device is greater than the threshold, the system 100 may control the ultrasound device to perform the execution function corresponding to the fingerprint information (e.g., the second fingerprint information). For example, as shown in FIG. 9 (a), if a pressure applied by the right index finger of the user on the collection device is below the threshold, a screen displays “Ultrasound Measurement” to remind the user that the current fingerprint corresponds to the ultrasound measurement function, preventing the user from making mistakes due to forgetting the mapping relationship; as shown in FIG. 9(b), if the pressure applied by the right index finger of the user on the collection device is above the threshold, the ultrasound measurement function is executed.

In some embodiments, different functions may be executed and/or different interfaces may be displayed based on pressing parameters of multiple fingers and corresponding thresholds. The multiple fingers may have various combinations, such as a combination of two fingers, a combination of three fingers, a combination of four fingers, etc. The pressing parameters of the multiple fingers may also vary, including simultaneous pressing strength, individual pressing strengths, sequential pressing order, and a count of presses for each finger, etc. The thresholds may be set based on the pressing parameters of the multiple fingers. In some embodiments, in response to a determination that at least one pressing parameter of the multiple fingers of the user on the collection device is below the corresponding threshold, the execution function corresponding to the fingerprint information may be displayed to the user; in response to a determination that the at least one pressing parameter of the multiple fingers of the user on the collection device is above the corresponding threshold, the execution function corresponding to the fingerprint information may be performed. For example, if a pressure applied by the left index finger and the left middle finger of the user on the collection device is below the corresponding threshold, the screen displays “Interface Switching” to remind the user that the current fingerprints corresponds to the “Interface Switching” function; if the pressure applied by the left index finger and the left middle finger of the user on the collection device is above the corresponding threshold, the interface switching function is executed.

By executing different functions and/or displaying different interfaces based on the pressing parameter(s) and the threshold(s) of one or more fingers, the user can be reminded of the function(s) corresponding to fingerprint(s) and also the issue of single functionality can be address. Prompting the user about the corresponding function(s) of the fingerprint(s) can enhance user experience, while using the pressing parameter(s) and the threshold(s) of one or more fingers can provide various implementation options for various functions, making it convenient for the user to use the ultrasound device efficiently.

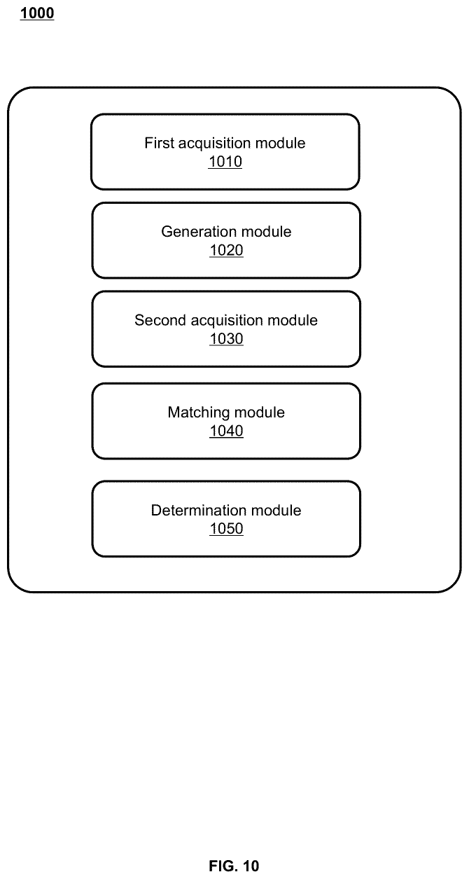

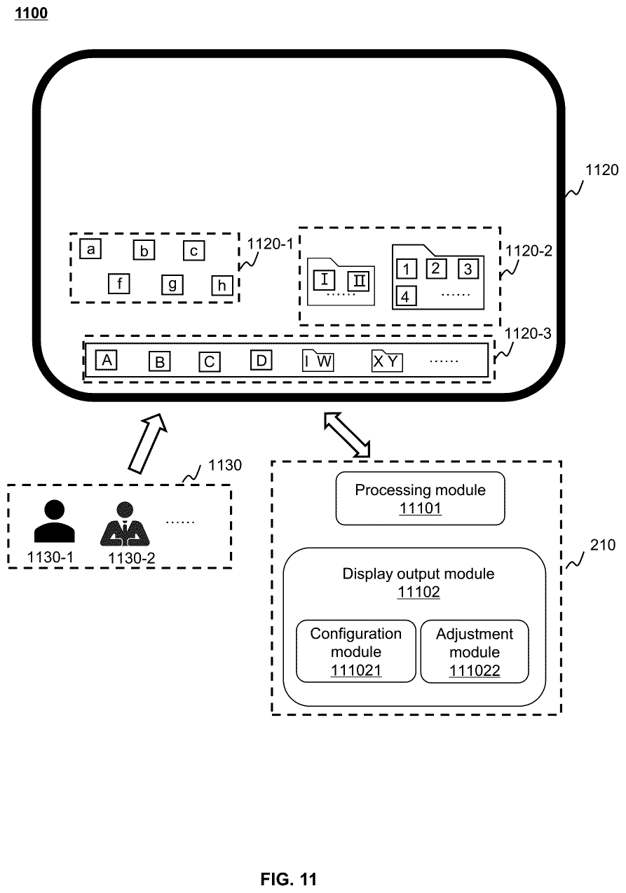

FIG. 10 is a block diagram illustrating an exemplary system for controlling a medical imaging device according to some embodiments of the present disclosure. As shown in FIG. 10, the system 1000 may include a first acquisition module 1010, a generation module 1020, a second acquisition module 1030, a matching module 1040, and a determination module 1050.

In some embodiments, the first acquisition module 1010 may be configured to obtain first fingerprint information of a user through a collection device.

In some embodiments, the generation module 1020 may be configured to configure an execution function corresponding to the first fingerprint information of the medical imaging device (e.g., an ultrasound device) and generate a first mapping relationship between the first fingerprint information and the execution function, wherein the execution function includes at least one of ultrasound image optimization, ultrasound measurement, interface switching, ultrasound image annotation, coded harmonic ultrasound imaging, scanning guidance page displaying, ultrasound probe switching, ultrasound clinical application, patient registration and management, ultrasound reporting, and ultrasound image reviewing.

In some embodiments, the second acquisition module 1030 may be configured to obtain second fingerprint information of the user through the collection device.

In some embodiments, the matching module 1040 may be configured to match the second fingerprint information with the first fingerprint information and obtain a matching result.

In some embodiments, the determination module 1050 may be configured to determine the execution function corresponding to the second fingerprint information based on a first mapping relationship and the matching result, and control the ultrasound device to implement an operation related to the execution function corresponding to the second fingerprint information.

In some embodiments, the system 1000 may also include a recognition unit. The recognition unit may be configured to identify information collected by a collection unit and/or information collected by a sensor unit. For example, the recognition unit may identify fingerprint information, palm print information, a protrusion on a surface of a glove, a pattern of a prosthesis, an electroencephalogram (EEG) wave, iris information, etc., collected by the collection unit. In some embodiments, the recognition unit may use one or more recognition techniques to identify the feature information (e.g., a region of interest where an effective fingerprint is located, a fingerprint feature, a palm print feature, a waveform feature of the EEG wave, an eye movement mode reflected by the iris information) in the information collected by the collection unit and/or the sensor unit. The recognition techniques may include a pattern recognition technique. The pattern recognition technique may include image processing techniques such as image filtering and image segmentation. In some embodiments, the recognition unit may communicate with the collection unit and/or the sensor unit through an interface (e.g., a USB interface). In some embodiments, the recognition unit may be part of the collection device.

It should be noted that the above descriptions of the various modules are for convenience and do not limit the scope of the present disclosure. It may be understood that for those skilled in the art, after understanding the principles of the system 1000, various combinations of the modules may be arbitrarily made, or constitute subsystems connected to other modules. In some embodiments, the second acquisition module 1030 and the matching module 1040 disclosed in FIG. 10 may be different modules in the system 1000, or they may be a single module implementing the functions of the two or more modules mentioned above. For example, the various modules may share a storage module, or each module may have its own storage module. Such variations are within the scope of protection of the present disclosure.

FIG. 11 is a schematic diagram illustrating an exemplary system for controlling an interface of a medical imaging device according to some embodiments of the present disclosure.

As shown in FIG. 11, in some embodiments, a system 1100 for controlling an interface of a medical imaging device (hereinafter referred to as the system 1100) may include a processor 1110, a display device 1120, and an operator 1130.

In some embodiments, the processor 1110 may be configured to control the medical imaging device to perform an operation. In some embodiments, the processor 1110 may correspond to the processing device 150.

In some embodiments, the processor 1110 may include one or more modules. In some embodiments, the one or more modules may include a processing module 11101 and a display output module 11102. In some embodiments, the display output module 11102 may include a configuration module 111021 and an adjustment module 111022.

The processing module 11101 may be configured to control the medical imaging device. In some embodiments, the processing module 11101 may receive an operation performed by the operator on an interface component. In some embodiments, based on a received operation, the processing module 11101 may control the medical imaging device.

The display output module 11102 may be configured to display a user interface including multiple types of interface components on the display device. The interface components may correspond to one or more functions associated with the medical imaging device and operations performed on the medical imaging device by the operator.

A function of the medical imaging device refers to an operation that the medical imaging device can perform, such as scanning a human body, capturing a medical image of the human body, etc. An operation performed on the medical imaging device refers to an operation performed by the operator on the medical imaging device, such as setting a scanning parameter, registering a scanning position with a position of the medical imaging device, etc. In some embodiments, based on an operation performed by the operator on an interface component, a corresponding function of the medical imaging device may be called, or a corresponding operation on the medical imaging device may be performed.

In some embodiments, the configuration module 111021 may be configured to determine a target interface component displayed in an initial layout based on a preset rule. In some embodiments, the configuration module 111021 may be configured to determine the layout of the target interface component on the user interface based on the preset rule.

The adjustment module 111022 may be configured to adjust the interface component (e.g., a position, a size, etc.) on the user interface.

The display device 1120 is configured to display the user interface including multiple interface components. In some embodiments, the user interface may span multiple display screens, composed of interfaces on the multiple display screens together.

In some embodiments, the display device 1120 may include an input device (e.g., the input device 140).

In some embodiments, the display device 1120 may receive the operation performed by the operator on the interface component. In some embodiments, the display device 1120 may send the operation performed by the operator on the interface component to the processor 1110.

In some embodiments, the display device 1120 may display the user interface including the multiple interface components by receiving user interface data from the display output module 11102.

An interface component refers to a component used to compose the user interface and perform a corresponding function. In some embodiments, the multiple interface components may include an application 1120-1, an application widget 1120-2, a program dock 1120-3, or a combination thereof.

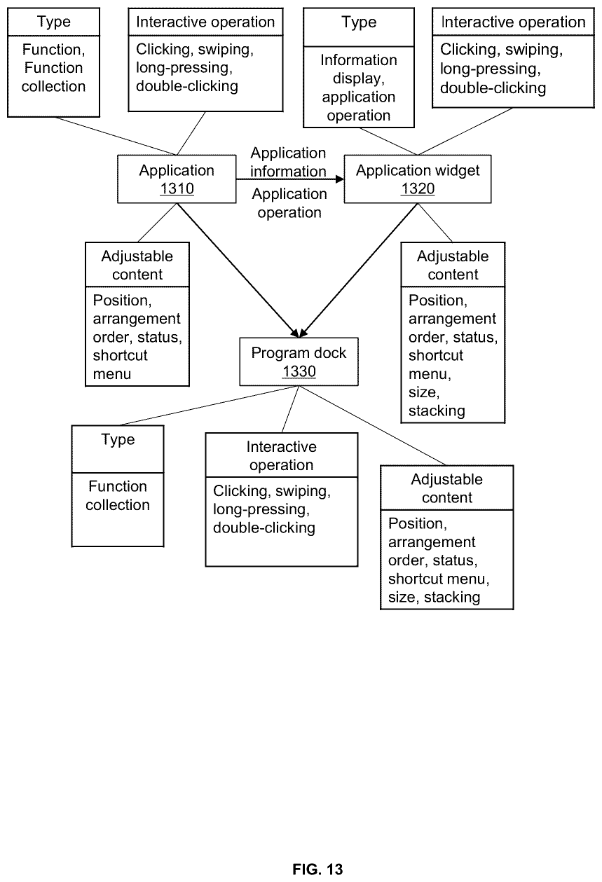

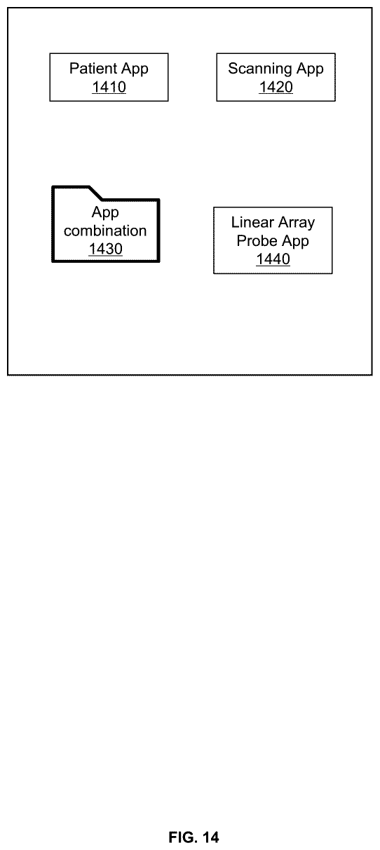

The application 1120-1 may correspond to various applications (apps). For example, different functions in the medical imaging device may correspond to different apps. The application 1120-1 may include various types of applications, for example, App a, App b, App c, App f, App g, App h, etc. By way of example, App a may be a Probe App, App c may be a Scan App, App g may be a Measurement App, App h may be a Report App, etc. In some embodiments, the application 1120-1 may also include applications for one or more key functions of the medical imaging device, such as a Convex Probe App, Cardiac Auto-Measurement App, etc. The application 1120-1 may extend beyond the above examples, and the mentioned applications are illustrative rather than restrictive. More descriptions regarding the application 1120-1 may be found in FIGS. 13 and 14 and the relevant descriptions thereof, which are not repeated here.

In some embodiments, the application widget 1120-2 refers to a component composed of one or more apps. The application widget 1120-2 may include various combinations of different applications. For example, the application widget 1120-2 may include two apps (App I, App II). As another example, the application widget 1120-2 may include four apps (App 1, App 2, App 3, App 4). By way of example, App I may be a Patient App, and App II may be a Linear Array Probe App. In some embodiments, the application widget 1120-2 may include a widget for one of the functions of the medical imaging device. For example, the application widget 1120-2 may include a Phased Array Probe Widget and a Heart Automatic Measurement Widget. The Phased Array Probe Widget may include multiple preset probes. The Heart Automatic Measurement Widget may include different measurement items. A count of applications included in the application widget 1120-2 may be set according to requirements. More descriptions regarding the application widgets 1120-2 may be found in FIGS. 13, 16, 17, 18 and the relevant descriptions thereof.

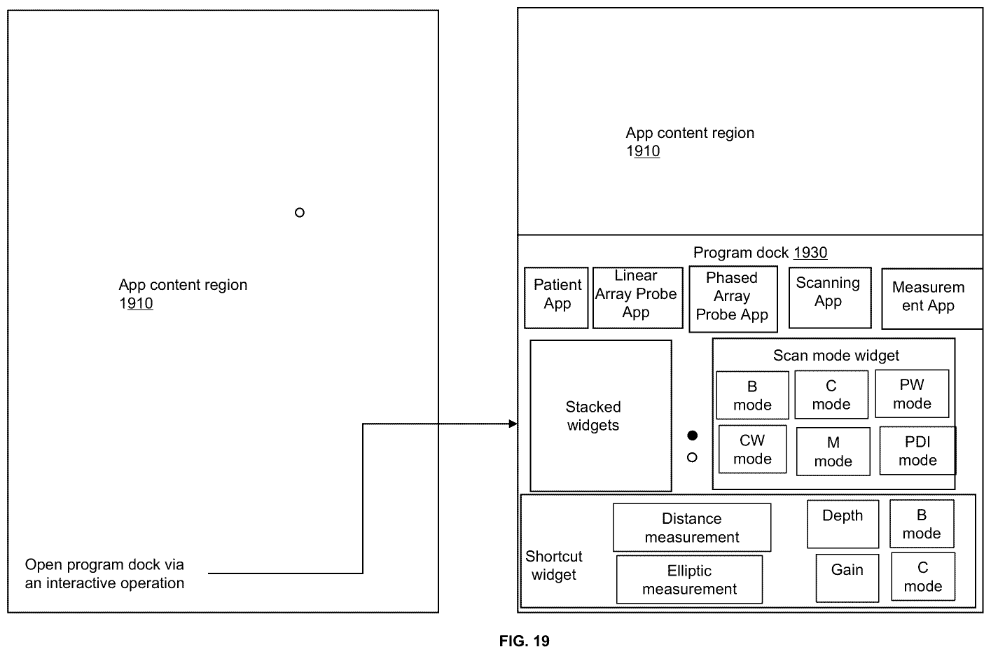

In some embodiments, the interface component may take the form of the program dock 1120-3. The program dock 1120-3 may include various combinations of different applications. For example, the program dock 1120-3 may include four apps (App A, App B, App C, App D) and two application widgets (I, W) and (X, Y), etc. The different representations (uppercase letters, lowercase letters, or numbers) of the above applications are merely illustrative and may represent the same or different applications, and are not limiting. More descriptions regarding the program dock 1120-3 may be found in FIG. 19 and the relevant descriptions thereof.

In some embodiments, the interface components are configured to correspond to the functions of the medical imaging device and operations of the user on the medical imaging device. This allows for quick invocation of the functions of the medical imaging device, facilitating convenient operation of the medical imaging device, reducing unnecessary steps and operations, and improving an efficiency of medical examinations and other work. In addition, the configuration of the interface components also expands an operational space for the user, improves a utilization of screen space, and enhances a flexibility and adaptability of the user interface to meet different user needs.

In some embodiments, the display device 1120 may be further configured to recognize second fingerprint information of the user, and the processor 1110 may be further configured to perform at least one of the following operations: executing a corresponding function or a corresponding operation based on the second fingerprint information; determining a user level and/or a user permission based on the second fingerprint information; executing a corresponding function and/or a corresponding operation based on user level and/or the user permission.

In some embodiments, a fingerprint region may be set on a touchable display device (e.g., a touchscreen). The fingerprint region may be set based on experience, requirements, and/or preferences. For example, the fingerprint region may be set in a bottom right corner of the touchscreen with minimal impact on detecting image displays. As another example, based on the user's preference, the fingerprint region may be set in a top left corner of the touchscreen. In some embodiments, the display device 1120 may be configured to recognize fingerprint information of the user, such as recognizing a finger pressing operation in the fingerprint region.

In some embodiments, the processor 1110 may execute the corresponding function or operation based on the second fingerprint information. For example, the processor 1110 may match the second fingerprint information with first fingerprint information to obtain a matching result. Based on a first mapping relationship and the matching result, the processor 1110 may determine the execution function corresponding to the second fingerprint information and control the medical imaging device to perform an operation related to the execution function.

In some embodiments, the function or operation corresponding to fingerprint information include functions or operations corresponding to applications, application widgets, and/or functions or operations included in the program dock.

In some embodiments, the medical imaging device includes an ultrasound device, and the function or operation corresponding to fingerprint information includes at least one of ultrasound image optimization, ultrasound measurement, interface switching, ultrasound image annotation, coded harmonic ultrasound imaging, scanning guidance page display, ultrasound probe switching, ultrasound clinical application, patient registration and management, ultrasound reporting, and ultrasound image reviewing.

In some embodiments, the processor 1110 may match the second fingerprint information with the first fingerprint information to obtain a matching result. Based on a third mapping relationship and the matching result, the processor may determine the user level and/or the user permission corresponding to the second fingerprint information.

The third mapping relationship may represent a correspondence between the first fingerprint information and the user level and/or the user permission. In some embodiments, the first fingerprint information may include fingerprint information of multiple fingers of the user. For example, the first fingerprint information may include ten fingerprint images corresponding to ten fingers of the user. In some embodiments, the third mapping relationship may include a correspondence between a single finger's fingerprint image (e.g., a fingerprint image of the right index finger) and the execution function, or a correspondence between a combination of two or more fingerprint images of the user's fingers and the execution function. For example, the third mapping relationship may include a correspondence between a combination of fingerprint images of two fingers (e.g., the index finger and the middle finger of the left hand) of the user, and the user level and/or the user permission. In this way, when the system 1100 recognizes the combination of the fingerprint images of the two fingers, the system automatically identifies the user level and/or the user permission, or the system 1100 further executes the corresponding function and/or operation based on user level and/or the user permission.

In some embodiments, after the user completes the setup, the processor 1110 may store the fingerprint information of the user who completes the setup, along with the user level and/or the user permission corresponding to the user. For example, the processor 1110 may package the fingerprint information of the user into personal fingerprint information of the user and store the personal fingerprint information in a personal fingerprint database of the user. In some embodiments, after the processor 1110 completes the packaging and storage of the fingerprint information of the user, the system 1100 may match the fingerprint information with the personal fingerprint information of the user when recognizing fingerprint information of the user in the future.

In some embodiments, the operator 1130 may be a user of the medical imaging device, such as a system administrator 1130-1, a doctor 1130-2, etc.

In some embodiments, the system 1100 may further include a collection device (not shown in the drawings). The collection device may be configured to obtain the second fingerprint information of the user. The processor 1110 may be further configured to perform at least one of the following operations: executing a corresponding function or a corresponding based on the second fingerprint information; determining the user level and/or the user permission based on the second fingerprint information; and executing a corresponding function and/or a corresponding operation based on the user level and/or the user permission.

In some embodiments, the collection device may include a touchpad, a touchscreen, one or more physical buttons (e.g., one or more physical buttons set on the terminal 130, the input device 140, etc.).

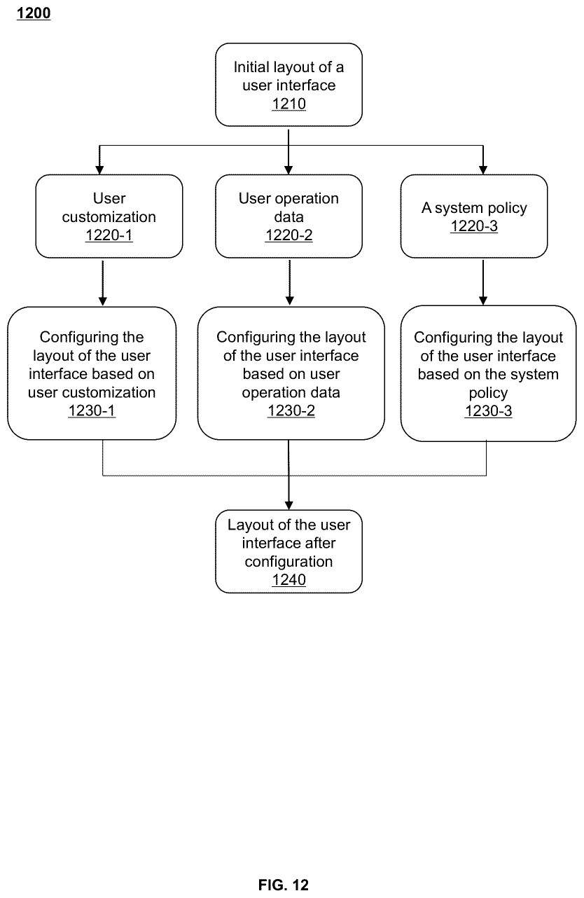

FIG. 12 is a flowchart illustrating an exemplary process for configuring a user interface according to some embodiments of the present disclosure.

In some embodiments, an interface component may be obtained by configuring an initial layout of the user interface based on a preset rule.

In some embodiments, when configuring the user interface, an initial layout 1210 of the user interface is first obtained. Subsequently, the preset rule is acquired, and the user interface is then configured based on the preset rule, resulting in a configured user interface 1240.

The initial layout of the user interface refers to an initial state of the user interface. In some embodiments, the initial layout of the user interface may be a default layout of the user interface, such as default settings at a factory, in which positions of various apps or functional controls are predefined by a manufacturer.

The preset rule refers to a rule used for laying out the user interface. In some embodiments, the preset rule may include at least one of user customization 1220-1, determined based on user operation data 1220-2 or based on a system policy 1220-3. In some embodiments, the preset rule may also include other types, such as a user fingerprint information-based rule.

The user customization refers to an autonomous adjustment made by a user as needed, such as an adjustment on positions and orders of apps.

In some embodiments, when the preset rule is user customization 1220-1, the layout of the user interface is configured based on the user customization 1220-1 through operation 1230-1. In some embodiments, operation 1230-1 may be performed by the configuration module 111021.

In some embodiments, an editing state of the user interface may be activated by the user, and by performing specified operations, such as dragging app icons, the configuration module 111021 may dynamically store location information of the apps in real time.

The preset rule determined based on operation data refers to a rule that an adjustment is made based on user operation data, such as a role of the user, historical data of an operation, etc. In some embodiments, the apps may be automatically adjusted based on the role of the user, a scanned region, etc.

In some embodiments, when the preset rule is based on operation data 1220-2, the layout of the user interface is configured based on user operation data 1220-2 through operation 1230-2. In some embodiments, operation 1230-2 may be performed by the configuration module 111021.

In some embodiments, the role of the user may be set. For example, the role of the user may be set by a system administrator, where the system administrator may include a service engineer, a department head, etc. As another example, the role of the user may be set by the user during account registration. In some embodiments, the role of the user may be categorized based on different requirements. For example, the role of the user may be categorized based on a doctor's position into a chief doctor, an attending physician, an intern, etc. As another example, the role of the user may be categorized based on specialties into a cardiology specialist, an obstetrics specialist, etc.

In some embodiments, the configuration module 111021 may automatically adjust the apps based on the role of the user. For example, if the user is a doctor specializing in cardiology, the configuration module 111021 may place a Phased-array Probe App commonly used by cardiologists in the first app position. In some embodiments, the user may choose whether to make an adjustment based on user operation data 1220-2 according to needs.

In some embodiments, the configuration module 111021 may automatically adjust the apps based on the scanned region. For example, if the scanned region is the abdominal organs, doctors typically use a convex array probe for abdominal organ scanning. The configuration module 111021 may prompt the user to select the convex array probe for scanning by configuring a small flashing icon on an application for the convex array probe.

The preset rule determined based on a system policy refers to a rule that an automatic adjustment of the apps is made by a system based on a product operation strategy, such as a policy updated after a system upgrade.

In some embodiments, when the preset rule is based on a system policy 1220-3, the layout of the user interface is configured based on a system policy 1220-3 through operation 1230-3. In some embodiments, operation 1230-3 may be performed by the configuration module 111021.

In some embodiments, the product operation strategy may be synchronized with the system through an online manner or an offline manner. The online manner includes Over-The-Air (OTA) updates, and the offline manner includes importing the product operation strategy through an external medium (e.g., a USB drive) into the system. The product operational strategy may include at least one of a target app to be adjusted, a target position, an effective time, an expiration time, etc. In some embodiments, the system administrator or the user may choose whether to adjust based on a system policy 1220-3 according to needs.

The user fingerprint information-based rule refers to a rule that an automatic adjustment of the user interface component is made based on fingerprint information collected by a collection device. For example, the user fingerprint information-based rule may include recognizing the role of the user based on the fingerprint information, and displaying the initial layout of the interface component corresponding to the role of the user after the user logs in for the first time using the fingerprint information (e.g., pressing the Home button with the index finger, long-pressing the fingerprint region on a touchpad and/or a touchscreen with an index finger, etc.). As another example, the user fingerprint information-based rule may include real-time storage of a user-initiated custom operation that modifies (e.g., dragging, hiding, adding to the program dock, etc.) the interface component, which allows the display of the altered interface component during subsequent use and/or upon logging in again with the fingerprint information.

The fingerprint region refers to a region on the touchscreen used to obtain the fingerprint information. The fingerprint region may be set based on experience, requirements, and/or preferences. For example, the fingerprint region may be set in a lower right corner of the touchscreen with minimal impact on detecting image display. As another example, the fingerprint region may be set in an upper left corner of the touchscreen based on the user's preference.

In some embodiments, the fingerprint information may be obtained through one or more physical buttons, such as one or more physical buttons on the terminal 130, the input device 140, etc.

Configuring the initial layout of the user interface based on the preset rule according to some embodiments of the present disclosure can enhance a measurement efficiency of the user. For example, upgrading the functionality of an automatic heart measurement application through a system upgrade can effectively improve the measurement efficiency of the user by automatically placing the automatic heart measurement application in the first app position through adjusting system policies.

FIG. 13 is a schematic diagram illustrating exemplary interface components according to some embodiments of the present disclosure.

The interface components may include various types, such as an application (App), an application widget, a program dock, etc. In some embodiments, the interface components on the user interface may include one or more of an application (App) 1310, an application widget 1320, or a program dock 1330.

A main screen of the user interface refers to a default displayed screen, such as Home or homepage. In some embodiments, the main screen of the user interface may include at least one App 1310 and at least one application widget 1320.

An application screen of the user interface refers to an application interface displayed when using an application. In some embodiments, the application screen of the user interface may include the program dock 1330. In some embodiments, the program dock 1330 may be included in the main screen of the user interface.

In some embodiments, the App 1310 may correspond to at least one function of one or more functions associated with a medical imaging device to perform an operation on a medical imaging device and/or an operation related to medical imaging. In some embodiments, the App 1310 may correspond to some main functions of the medical imaging device, such as patient, probe, scanning, measurement, annotation, reporting, etc. In some embodiments, the App 1310 may correspond to a key function or a set of key functions, such as convex array probe application, automatic heart measurement application. In some embodiments, the App 1310 may also correspond to a collection of other functions, such as notification and configuration. In some embodiments, the App 1310 may correspond to processing of a medical image, such as three-dimensional (3D) model generation, image annotation, etc.

In some embodiments, the application widget 1320 may correspond to the App 1310 to achieve extended functions related to the App 1310, such as displaying application information, invoking in-app functions, etc. In some embodiments, the application widget 1320 may correspond to at least one piece of information or operation related to the App 1310. In some embodiments, the application widget 1320 may include one or more internal elements, each of the one or more internal elements corresponding to application information or a function, which may come from one or more Apps.

In some embodiments, the program dock 1330 may include one or more Apps and/or application widgets to achieve functions related to the Apps. In some embodiments, the program dock may be displayed in an interface corresponding to the Apps by invocation. Specified operations may be achieved by calling the Apps or application widgets in the program dock.

The interface components may perform invoking or implement related functions through interactive operations. In some embodiments, interface components may be represented as icons, corresponding to one or more interactive operations, such as clicking, swiping, long-pressing, double-clicking, etc. In some embodiments, different interactive operations may correspond to different functions, for example, clicking corresponds to selection, long-pressing brings up a shortcut menu, etc. In some embodiments, a same interactive operation may correspond to different functions for different interface components, for example, clicking an App may open a corresponding functional interface, while clicking an application widget may display contents of the widget. In some embodiments, the user may define the functions corresponding to interactive operations, for example, defining clicking an App as selection and defining double-clicking the App as opening.

Each type of interface components may be further classified according to specific criteria, and the classification criteria may be determined based on a specific type of the interface components. For example, the interface components may be classified based on whether they can be executed to be classified, a count of included elements, etc.

The interface component may be adjusted, for example, in terms of size, position, color, content, corresponding function, etc. In some embodiments, the interface component on the user interface is configured to be adjustable based on a user operation to meet the user's needs. In some embodiments, the user may interact with the interface component to make an adjustment, for example, adjusting a position and/or order of the interface component by dragging. In some embodiments, the adjustment to the interface component may also be made in various other ways, such as eye movement control, brain control, gesture control, voice control, etc.

In some embodiments, the adjustment to the interface component may include an adjustment on an appearance, a position, a corresponding function, etc., of the interface component. Different interface components may include different adjustable contents. For example, an App may not include an adjustment on a size. In some embodiments, the adjustment to the interface component may span multiple screens, for example, dragging the interface component from screen A to screen B.

In some embodiments, the adjustment to the interface component may be initiated by the user. For example, the user may place a most frequently used interface component in an easily accessible position. As another example, the user may add or remove content included in the interface component as needed.

In some embodiments, the adjustment to the interface component may be made automatically based on user operation data. For example, based on historical operation data of the user, an interface component that is most frequently used by the user may be placed in a position that is most easily seen and operated (e.g., a center of the interface). As another example, based on the historical operation data of the user, an interface component with a lower usage frequency may be placed in a relatively inconspicuous position (e.g., near an edge of the interface), or even folded to reduce space occupation. As yet another example, when a frequency of invoking a certain interface component reaches a threshold, an arrangement order for the interface component is moved forward by one position.

In some embodiments, the adjustment to the interface component may be made automatically based on a user level. For example, the user level may include a hospital level, a department level, a doctor level, etc., wherein the hospital level is higher than the department level, and the department level is higher than the doctor level. After adjusting an interface component for a higher-level user (e.g., a hospital-level user), an interface component corresponding to a lower-level user affiliated to the higher-level user is automatically adjusted accordingly.

In some embodiments, the adjustment to the interface component may be made automatically based on a user permission. For example, in some embodiments, the user permission may include a management permission, a usage permission, a read-only permission, a trial permission, a maintenance permission, etc. For a user with the management permission, interface components related to user management, role management, etc., may be highlighted. For a user with the read-only permission, customization of widgets may not be allowed. For a user with the trial permission, the interface component may only display an application and/or a widget related to a new feature and/or a new application, hiding other applications and/or widgets.

In some embodiments, the adjustment to interface component may also be made through other means, for example, through self-adaptation.

As shown in FIG. 13, in some embodiments, the App 1310 may be classified, based on a count of corresponding contents, into different types such as function, function collection, etc. For example, the App 1310 may correspond to a specific function in the medical imaging device, such as probe, scanning, measurement, etc. As another example, the App 1310 may correspond to a collection of multiple functions in the medical imaging device, such as a convex array probe application, configuration, etc.

FIG. 14 is a schematic diagram illustrating exemplary applications according to some embodiments of the present disclosure.

As shown in FIG. 14, in some embodiments, the Apps may correspond to main functions of a medical imaging device, such as a Patient App 1410, a Scanning App 1420; or the Apps may correspond to a key function or a collection of key functions in the main functions, such as a Linear Array Probe App 1440.

In some embodiments, the Apps may be a combination of multiple Apps represented in a folder form, for example, an App combination 1430.

As shown in FIG. 13, in some embodiments, interactive operations corresponding to the App 1310 may include clicking, swiping, long-pressing, double-clicking, etc. In some embodiments, clicking an App icon may enter a corresponding functional interface of the App, and long-pressing the App icon may display a shortcut menu for the App and a main screen.

In some embodiments, the interactive operations with Apps may also include various other forms, such as eye movement control, brain control, gesture control, voice control, etc.

The shortcut menu is used for quick access to functions. The shortcut menu for an App may be used for quick access to the functions of the App. The shortcut menu on the main screen may be used for quick access to system functions. In some embodiments, distinctions may be made between different types of shortcut menus, such as dividing lines, dividing icons, etc., to help a user better distinguish between Apps and shortcut menus on the main screen.

As shown in FIG. 13, in some embodiments, adjustable contents for the App 1310 may include: a position, an arrangement order, a status, a shortcut menu, etc., of the App 1310. The position of the App 1310 is a position of an icon of the App 1310 on a screen. The arrangement order of the App 1310 is a spatial arrangement order of icons of the App 1310 on the screen, i.e., the relative positions of the icons of the App 1310 on the screen, such as left-right, up-down, etc.

In some embodiments, the adjustable contents for the App 1310 may also include splitting and restructuring. Specifically, if an App corresponds to more than one function, the App may be split based on the functions or function combinations, and the splitting functions and combinations may correspond to new Apps, which may be combined with other Apps.

In some embodiments, the decision to split an App may be based on a usage frequency of the App. In some embodiments, if the usage frequency of a function exceeds a threshold, the system 1100 may inquire the user whether the user wants to split the App, or the system 1100 may automatically split the App and notify the user.

The status of the App 1310 is used to prompt a user action or explain a situation of the App 1310. In some embodiments, a Measurement App may use small icons to indicate a count of measurements the user needs to perform. In some embodiments, a Probe App may use a flashing icon or color changes to inform the user to select the specific probe corresponding to the Probe App.

In some embodiments, adjustments may be made to items included in the shortcut menu of the App 1310, such as adjusting the order, adding or removing items, etc.

In some embodiments, at least one of the position, the arrangement order, the status, the shortcut menu, etc., of the App 1310 may be manually adjusted by the user or automatically adjusted by the system 1100 for the App 1310 to meet needs of the user.

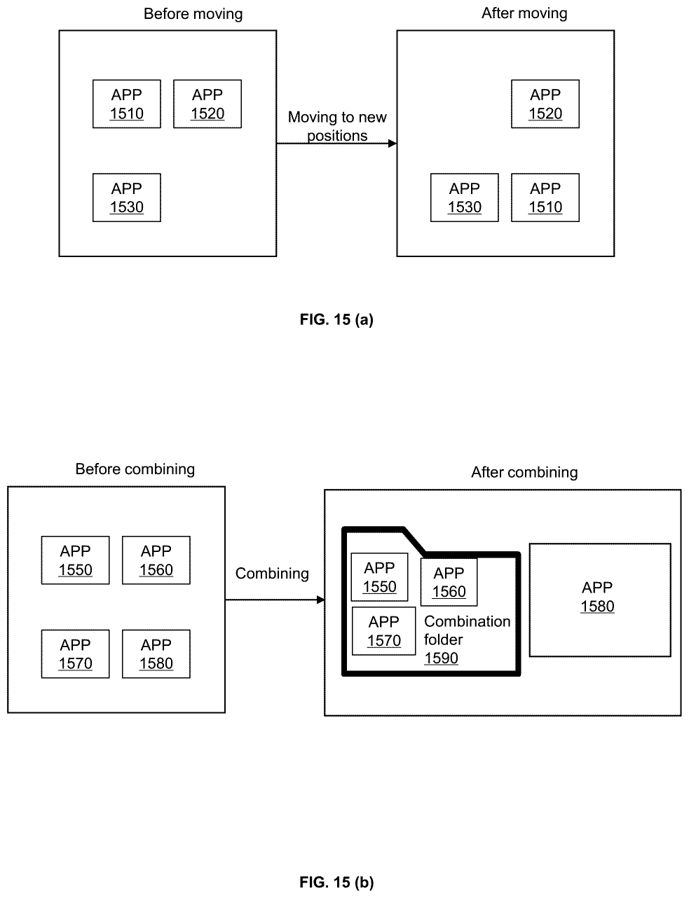

FIG. 15 (a) is a schematic diagram illustrating moving of an exemplary application according to some embodiments of the present disclosure.

In some embodiments, as shown in FIG. 15 (a), a user interface includes App 1510, App 1520, and App 1530. The App 1510 may be moved to a new position. Before the movement, the position of the App 1510 is on an upper left corner of a screen. The App 1510 may be moved in various manners, such as dragging the icon of the App 1510 to a new position. After the movement, the icon of the App 1510 is located on a lower right corner of the screen.

FIG. 15 (b) is a schematic diagram illustrating combining of exemplary applications according to some embodiments of the present disclosure.

In some embodiments, as shown in FIG. 15 (b), before combination, there are four independent Apps on the screen: App 1550, App 1560, App 1570, and App 1580. Multiple Apps may be combined into an App folder. After the combination, the screen includes a combination folder 1590 containing App 1550, App 1560, and App 1570, and App 1580.

As shown in FIG. 13, in some embodiments, the application widget 1320 may be classified into different types based on corresponding functions, such as information display, application operation, etc.

In some embodiments, the application widget 1320 may be configured to display information related to the App 1310. For example, a Measurement App may define a widget for displaying measurement results to provide the functionality of viewing measurement results. A Trackball App may define a widget for indicating trackball operations to inform the user of the current function of the trackball and its buttons.

In some embodiments, the application widget 1320 may be configured to directly invoke the functions related to the App 1310. For example, “Distance Measurement” in a measurement widget may activate a distance measurement tool.

In some embodiments, the application widget 1320 may be configured to open the interface of a corresponding application directly. For example, “Enter Regular Measurement” in the measurement widget may enter the Measurement App and locate to an application interface for regular measurements.

In some embodiments, the user may add and/or remove widget(s). For example, the user may select one or more applications simultaneously, long-press the selected one or more applications to get a right-click menu, and set widget(s) related to the one or more applications through the right-click menu. For example, the user may long-press a Scan Parameter App to get a right-click menu and set a widget related to the Scan Parameter App through the right-click menu.

In some embodiments, the user and/or a processor may add and/or remove widget(s) based on experience and/or requirements. For example, based on pages and settings of a Probe App, the Scan Parameter App, and the Measurement App, widget A corresponding to health examination package A (including thyroid ultrasound, lower abdominal ultrasound) may be added. Similarly, based on different pages and settings of the Probe App, the Scan Parameter App, and the Measurement App, widget B corresponding to health examination package B (including thyroid ultrasound, upper abdominal ultrasound, lower abdominal ultrasound) may be added. Likewise, based on different pages and settings of the Probe App, the Scan Parameter App, and the Measurement App, widget C corresponding to examination items for an emergency patient (including cardiac ultrasound, head ultrasound) may be added.

In some embodiments, different fingerprint information may correspond to different widgets. For example, the combination of the index finger and the middle finger of the left hand corresponds to running widget A, the combination of the index finger and the ring finger of the left hand corresponds to running widget B, and the combination of the left middle finger and ring finger corresponds to running widget C.

In some embodiments, the processor may adjust the positions of the widgets on a display screen as needed. For example, the processor may place widgets containing new applications and/or new features in a prominent position to guide the user to try out the new applications and/or features.

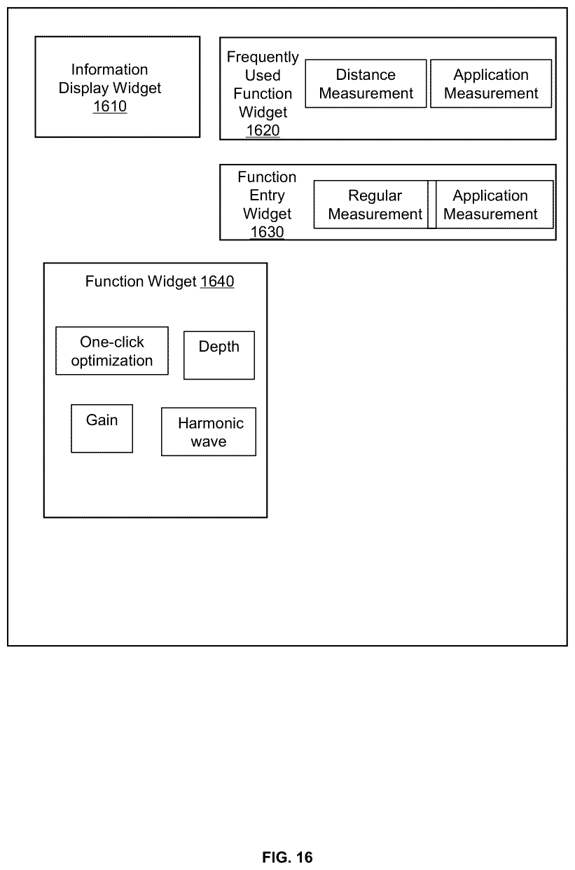

FIG. 16 is a schematic diagram illustrating exemplary application widgets according to some embodiments of the present disclosure.

As shown in FIG. 16, in some embodiments, the application widget 1320 may display application information, for example, Information Display Widget 1610; the application widget 1320 may directly perform operations related to the application, for example, a Frequently Used Function Widget 1620; the application widget 1320 may be an entrance to enter a corresponding functional interface, for example, a Function Entry Widget 1630; or the application widget 1320 may be a combination of the above multiple types, for example, a Function Widget 1640.

In some embodiments, application widgets may be linked to applications. For example, in a widget, an application currently running or interacting with the user may be made prominent to the user. As shown in FIG. 16, when the processor recognizes that a Routine Measurement App is running, the Function Entry Widget 1630 containing the Routine Measurement App is be made prominent to the user. An App may be made prominent to the user in various ways, for example, through highlighting, enlarging, flashing, etc.

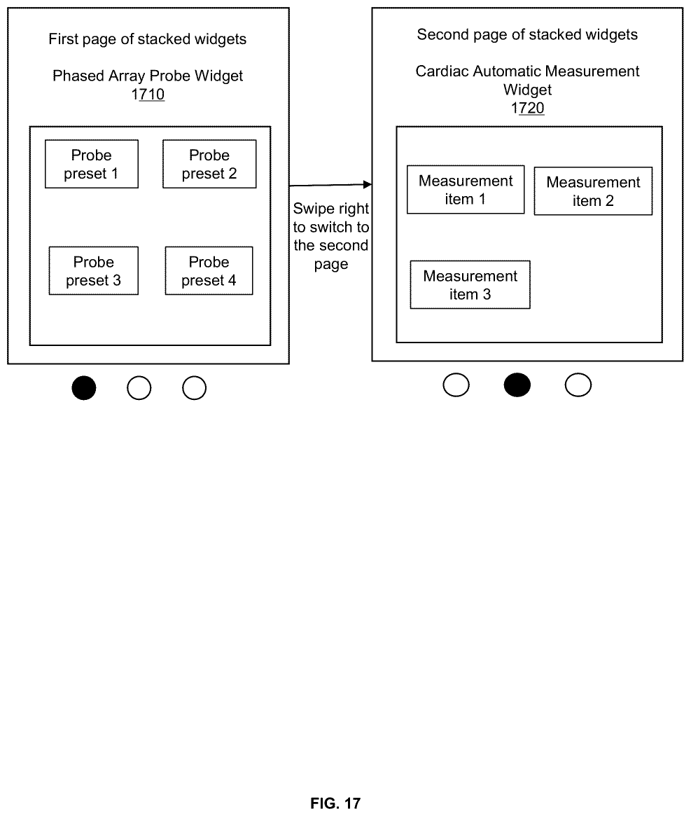

FIG. 17 is a schematic diagram illustrating exemplary stacked application widgets according to some embodiments of the present disclosure.

As shown in FIG. 17, the stacked application widgets include 3 pages, represented by circles at the bottom of the application widgets. The black circle represents a currently displayed page, and the white circles represent pages not currently displayed. The count of the circles represents the count of pages. In some embodiments, different pages may be switched by swiping left or right. As shown in FIG. 17, before switching, the displayed page is the first page of stacked widgets, i.e., a Phased Array Probe Widget 1710. Swiping to the right switches to the second page of the stacked widgets, i.e., a Cardiac Automatic Measurement Widget 1720.

As shown in FIG. 13, in some embodiments, the program dock 1330 may be a collection of functions, including at least one app and at least one application widget. In some embodiments, the interactive operations and adjustable contents corresponding to the program dock 1330 are similar to the interactive operations and adjustable contents corresponding to the App 1310 and the application widget 1320, as described above.

In some embodiments, the program dock 1330 may be activated within the display interface of the App 1310. In other words, the program dock 1330 is displayed within the display interface of the App 1310. In some embodiments, the program dock may be activated/deactivated through interactive operations, such as swiping up from a bottom of the screen to activate the program dock 1330 and swiping down to deactivate the program dock 1330.

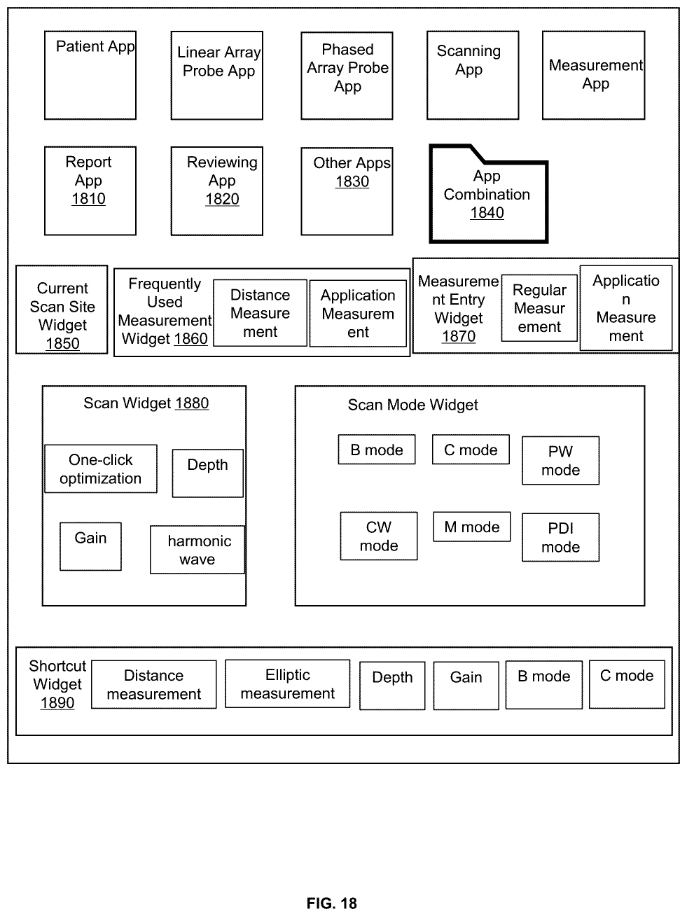

FIG. 18 is a schematic diagram illustrating exemplary interface components according to some embodiments of the present disclosure.

As shown in FIG. 18, a user interface includes various types of interface components, including apps such as a Report App 1810, a Review App 1820, other Apps 1830, an App Combination 1840, and interface widgets such as a Current Scan Site Widget 1850, a Frequently Used Measurement Widget 1860, a Measurement Entry Widget 1870, a Scan Widget 1880, a Shortcut Widget 1890, etc.

In some embodiments, an application widget may span multiple apps. In other words, functions of multiple apps may be freely combined into a single widget, for example, the Shortcut Widget 1890 shown in FIG. 18.

In some embodiments, interface components may correspond to a sequence of one or more functions associated with a medical imaging device executed in order. The interface components may include apps, application widgets, etc.

In some embodiments, one type of multiple types of the application widgets may correspond to a sequence of one or more functions associated with the medical imaging device executed in order, with each function in the sequence corresponding to a function associated with the medical imaging device. In some embodiments, multiple functions may be added to one application widget, and the multiple functions may come from a single application or different applications. Then the execution order of the multiple functions may be determined, or the multiple functions may be executed based on the order in which the multiple functions are added. For example, the user may add a common operational process for viewing cardiac blood flow during a heart scan to an application widget. The application widget includes the following operations in order: 1. Open B-mode; 2. Open C-mode; 3. Adjust a position and a size of a Region of Interest (ROI) under C-mode; 4. Open PW mode; 5. Freeze an image; 6. Measure PW envelope (or other); 7. Unfreeze the image, wherein B, C, PW, ROI adjustment, freeze/unfreeze, etc., may belong to a Scan App, while envelope measurement may belong to a Measurement App. The series of operations may be automatically executed in order by calling the application widget without the need for the user to manually switch between apps.

In some embodiments, the user may define a fixed, commonly used series of ordered operations using an application widget, greatly saving operation time, reducing user repetitive operations, reducing operation complexity, and alleviating user burden. In some embodiments, an experienced user may define a complex series of operations as an application widget, and other users may directly use the application widget to obtain a desired operation result without worrying about the specific operational process, saving training resources, reducing learning difficulty, and improving the user-friendliness of user interaction.

As shown in FIG. 13, in some embodiments, the interactive operations corresponding to the application widget 1320 may include clicking, swiping, long-pressing, double-clicking, etc. In some embodiments, for different types of application widgets, the same interactive operation may correspond to different functions. For example, clicking a Measurement Result Widget may display measurement results; clicking “Distance Measurement” in a Measurement Widget may activate or deactivate a distance measurement tool; clicking “Enter Routine Measurement” in the Measurement Widget may enter an application program interface of the Measurement App.

In some embodiments, long-pressing an application widget may activate a shortcut menu. The shortcut menu of the application widget is similar to the shortcut menu of the App 1310 as described above, and is not repeated here.

As shown in FIG. 13, in some embodiments, adjustable contents of the application widget 1320 may include: a position, an arrangement order, a status, the shortcut menu, a size, stacking, etc., of the application widget 1320. The adjustment of the position, the arrangement order, the status, the shortcut menu, etc., of the application widget 1320 is similar to that of the App 1310. More descriptions may be found in the above related descriptions.

The size of the application widget refers to a space that application widget occupies on a screen. In some embodiments, the system 1100 may provide application widgets of different sizes to include or display different counts of application functions and information simultaneously. In some embodiments, the user may modify the size of the application widget based on a space or arrangement of the home screen to make the application widget larger or smaller.

In some embodiments, the size of the application widget 1320 may be defined as a multiple of an app icon, for example, n′m app icons, wherein n and m denote positive integers determined based on a screen size. For example, the size of the application widget may be 1*1 app icon, 2*1 app icons, 2*2 app icons, 3*3 app icons, 4*4 app icons, etc. This ensures an orderly arrangement on the home screen, making full use of the screen space.

Stacking refers to placing multiple interface components in the same space on the screen and switching to display different interface components. In some embodiments, a plurality of application widgets may be stacked, i.e., the plurality of application widgets may be stacked in a same space, and switching between the plurality of application widgets may be achieved by swiping up/down/left/right, etc. In some embodiments, a count of stacked application widgets and a currently displayed widget may be indicated by icons. In some embodiments, the plurality of application widgets may be stacked through user operations or automatic adjustments by the system 1100.

FIG. 19 is a schematic diagram illustrating an exemplary program dock according to some embodiments of the present disclosure.

In some embodiments, as shown in FIG. 19, after opening an application corresponding to the App 1310, a current display region of a screen is occupied by an app content region 1910. A program dock 1930 may be activated by an interactive operation, and after activation, the program dock 1930 is displayed in a lower part of the screen, covering part of an original display region of an app content region 1910. In some embodiments, a user may use functions corresponding to various interface components in the program dock 1930 through interactive operations.

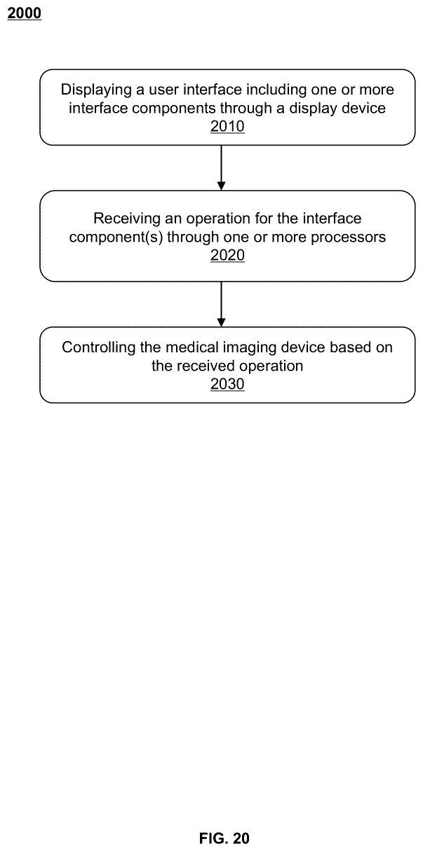

FIG. 20 is a flowchart illustrating an exemplary process for controlling an interface of a medical imaging device according to some embodiments of the present disclosure. As shown in FIG. 20, process 2000 includes the following operations:

In 2010, a user interface comprising multiple interface components may be displayed though a display device. In some embodiments, operation 2010 may be performed by the display output module 11102.

In some embodiments, the multiple interface components on the user interface may be configured to correspond to a function of the medical imaging device and an operation performed by the user on the medical imaging device. The function of the medical imaging device refers to an operation that the medical imaging device may perform on a medical examination subject, such as scanning, measurement, etc. The operation performed by the user on the medical imaging device refers to an operation performed on the medical device such as moving a probe, etc.

In some embodiments, the display output module 11102 may configure interface components based on functions of the medical imaging device. For example, the display output module 11102 may configure a scan mode component, a measurement component, a heart automatic measurement component, etc.

The operation performed by the user on the medical imaging device may include various operations. In some embodiments, the display output module 2102 may configure interface components based on the operation performed by the user on the medical imaging device. For example, the display output module 11102 may configure a common measurement component, etc.

In some embodiments, the interface components include one or more of an application, an application widget, or a program dock. More descriptions regarding the interface component may be found in FIGS. 14-19 and the related descriptions thereof.

In 2020, an operation performed by the user on the interface components may be received through one or more processors. In some embodiments, operation 2020 may be performed by the processing module 11101.

The operation performed by the user on the interface components refer to an interactive operation performed by the user on the interface components such as clicking, swiping, etc. In some embodiments, the operation performed by the user on the interface components may correspond to an instruction issued by the user, for example, an instruction for distance measurement, an instruction for heart automatic measurement, etc.

In some embodiments, the processing module 11101 may obtain fingerprint information, and the function of the medical imaging device or the operation associated with (e.g., performed on) the medical imaging device that correspond to the fingerprint information through a collection device or a display device. The fingerprint information corresponds to the function of the medical imaging device or the operation associated with (e.g., performed on) the medical imaging device, which may include at least one of ultrasound image optimization, ultrasound measurement, interface switching, ultrasound image annotation, coded harmonic ultrasound imaging, scanning guidance page displaying, ultrasound probe switching, ultrasound clinical application, patient registration and management, ultrasound reporting, ultrasound image reviewing, etc. For example, the processing module 11101 may obtain a fingerprint image, a pressing strength, a count of presses, etc. In some embodiments, the fingerprint information corresponds to the function of the medical imaging device or the operation associated with (e.g., performed on) the medical imaging device. For example, a combination of an index finger fingerprint image of the user and a pressing strength of 100N from the index finger may correspond to the image optimization function of the medical imaging device.

In 2030, the medical imaging device may be controlled based on the received operation. In some embodiments, operation 2030 may be performed by the processing module 11101.

In some embodiments, the processing module 11101 may control the medical imaging device to perform a corresponding measurement based on the received operation. For example, upon receiving an instruction for heart automatic measurement, the medical imaging device performs a measurement of the heart of a patient.

In some embodiments, the processing module 11101 may execute the function or operation corresponding to the fingerprint information through the one or more processor. For example, the processing module 11101 may execute the image optimization function corresponding to the fingerprint information.

It should be noted that the description of process 2000 above is for illustration and explanation purposes only and does not limit the scope of the present disclosure. Those skilled in the art may make various modifications and changes to process 2000 under the guidance of the present disclosure. However, these modifications and changes are still within the scope of the present disclosure. For example, in process 2000, it is possible to control the medical imaging device while receiving the operation performed by the user on the interface components.

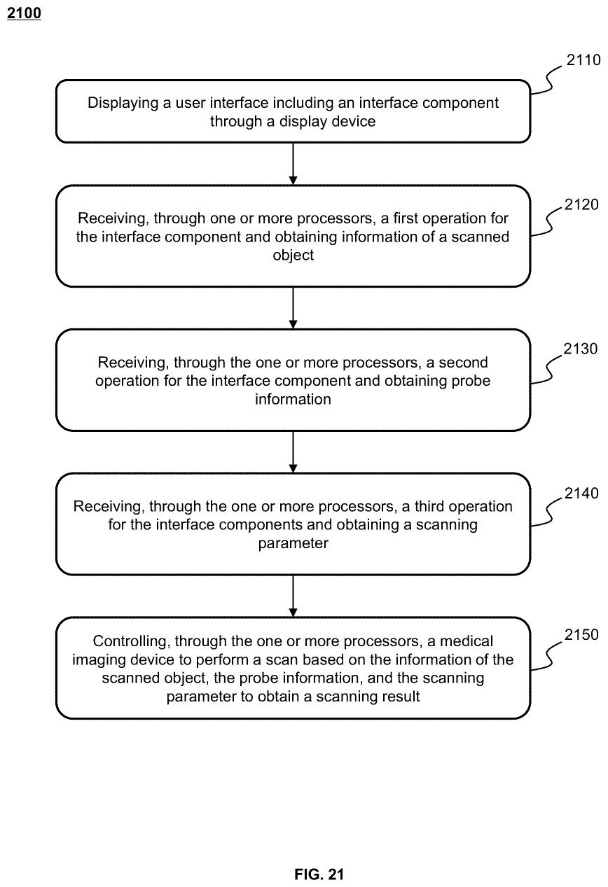

FIG. 21 is a flowchart illustrating an exemplary process for imaging according to some embodiments of the present disclosure.

As shown in FIG. 21, process 2100 includes the following operations. In some embodiments, process 2100 may be performed by the processing device 140.

In 2110, a user interface comprising an interface component may be displayed through a display device.

In some embodiments, the user interface may be adjusted based on a country and/or region where a medical imaging device is sold. For example, the user interface may be adjusted based on language, display conventions, etc., in different countries and/or regions. In some embodiments, the user interface may be adjusted based on a hospital, an organization, and/or a specific department using the medical imaging device. For example, functions and/or widgets related to generating detection reports may be hidden on the user interface for departments such as cardiology that rarely use this function.

In some embodiments, the user interface may be adjusted based on the identity and/or preferences of a user. For example, the same layout of the user interface may be displayed for doctors in the same department according to department rules. As another example, more content related to parameters may be displayed for users who prefer to adjust the parameters themselves.

In some embodiments, the user interface may include an interface component.

In some embodiments, interface component is configured to correspond to a function of the medical imaging device and/or an operation performed on the medical imaging device. A type of the interface component includes at least one of an application, an application widget, a program dock. For example, Application A corresponds to ultrasound image optimization, Application B corresponds to ultrasound measurement, Application C corresponds to ultrasound image annotation, Application D corresponds to coded harmonic ultrasound imaging, Application E corresponds to scanning guidance page displaying, etc. As another example, an ultrasound probe widget, a heart automatic measurement widget, etc., may be displayed on the user interface. As yet another example, a program dock may be displayed on the user interface, and the program dock may include one or more applications, widgets, etc., corresponding to the function of the medical imaging device and/or the operation performed on the medical imaging device.

In some embodiments, the processing device 140 may display the user interface comprising the interface component corresponding to fingerprint information through a display device. In some embodiments, the processing device 140 may identify the identity, a need, a level, a permission, etc., of the user based on fingerprint information of the user, and display the corresponding user interface based on the identity, a need, a level, a permission, etc., of the user. For example, when identified second fingerprint information (e.g., first press the right index finger, then press the right middle finger) corresponds to an emergency need of a doctor, the user interface primarily displays applications, widgets, and program docks related to the examination items of emergency patients. As another example, when the identified second fingerprint information (e.g., first press the right index finger, then press the right ring finger) corresponds to a routine physical examination need of a doctor, the user interface primarily displays applications, widgets, and program docks related to routine physical examination items. As yet another example, when the identified second fingerprint information (e.g., fingerprint information of an administrator) corresponds to an administrator permission, the user interface primarily displays applications, widgets, and program docks related to user management.

In some embodiments, interaction with the user may be achieved through the user interface. Taking a touchscreen display device as an example, the user may click, swipe, long-press, double-click, etc., on the touchscreen displaying the user interface to make the medical imaging device perform corresponding functions. As another example, the user may press one or more fingers on any region or a specific fingerprint region of the touchscreen to make the medical imaging device perform the function corresponding to the finger pressing operation.

In some embodiments, the one or more processors may obtain the fingerprint information through a collection device or the display device and perform corresponding operations or functions based on the fingerprint information. In some embodiments, the one or more processors may obtain fingerprint information corresponding to a finger pressing operation. For example, the one or more processors may obtain a fingerprint image, a pressing strength, a pressing duration, a pressing order, etc. In some embodiments, different fingerprint information, such as combinations of different fingerprint images, may correspond to different execution functions. For example, a combination of fingerprint images of an index finger and a middle finger correspond to the execution function of ultrasound image optimization, and a combination of fingerprint images of a middle finger and a ring finger correspond to the execution function of ultrasound measurement. In some embodiments, different arrangements of the same two or more fingerprint images may correspond to different execution functions. For example, for the same index finger and middle finger of the same user, the execution function corresponding to pressing the index finger first and then pressing the middle finger may be coded harmonic ultrasound imaging, and the execution function corresponding to pressing the middle finger first and then pressing the index finger may be ultrasound measurement.

In 2120, a first operation for the interface component may be received and information of a scanned object may be obtained through the one or more processors.

The first operation may include operations such as clicking, swiping, long-pressing, double-clicking, etc., on applications and/or widgets related to the information of the scanned object. The first operation may also include pressing operations with one or more fingers.

The information of the scanned object may include the name, height, weight, blood pressure, examination site, medical history, etc., of the scanned object.

In 2130, a second operation for the interface component may be received and probe information may be obtained through one or more processors.

The second operation may include operations such as clicking, swiping, long-pressing, double-clicking, etc., on applications and/or widgets related to the probe information. The second operation may also include pressing operations with one or more fingers.

The probe information may include a type, a status, etc., of a probe. The type of the probe may include a superficial probe, a convex array probe, a linear probe, etc. The status of the probe may include normal, abnormal, missing, etc.

In some embodiments, after receiving the second operation for the interface component, the one or more processors may determine the probe information based on the information (e.g., the examination site) of the scanned object.

In some embodiments, after receiving the second operation for the interface component, the one or more processors may directly determine the probe information based on the second operation. For example, if the user predefines that the combined fingerprint images of the ring finger and little finger of the right hand correspond to the selection of a linear probe, the one or more processors may directly choose the linear probe upon receiving the image combination.

In 2140, a third operation for the interface component may be received and a scanning parameter may be obtained through the one or more processors.

The third operation may include operations such as clicking, swiping, long-pressing, double-clicking, etc., on applications and/or widgets related to the scanning parameter. The third operation may also include pressing operations with one or more fingers.

The scanning parameter may include a frame rate, a pulse width, a pulse frequency, a damping, a time gain control, etc.

In some embodiments, after receiving the third operation for the interface component, the one or more processors may automatically set the scanning parameter based on the information of the scanned object and/or the probe information.

In some embodiments, after receiving the third operation for the interface component, the one or more processors may automatically set the scanning parameter based on the third operation. For example, if the user predefines that the combined fingerprint images of the index finger, the middle finger, and the ring finger of the right hand correspond to setting the scanning parameter suitable for pregnancy examinations, the one or more processors may directly set the scanning parameter upon receiving the image combination.

In 2150, the medical imaging device may be controlled to perform a scan based on the information of the scanned object, the probe information, the scanning parameter through one or more processors, and a scanning result may be obtained.

In some embodiments, process 2100 may further include at least one of the following operations:

A fourth operation for the interface component may be received through the one or more processors, and the scanning result may be measured to obtain a measurement result. A fifth operation for the interface component may be received through the one or more processors, and the measurement result may be annotated. A sixth operation for the interface component may be received through the one or more processors, and an examination report may be generated for the scanned object.

The fourth operation may include operations such as clicking, swiping, long-pressing, double-clicking, etc., on applications and/or widgets related to measuring the scanning result. The fourth operation may also include pressing operations with one or more fingers.

The measurement of the scanning result may include a distance measurement, an application measurement, a routine measurement, etc. For example, the measurement of the scanning result may include measuring a biparietal diameter, a femur length, etc., based on the scanning result related to a fetal examination.

The fifth operation may include operations such as clicking, swiping, long-pressing, double-clicking, etc., on applications and/or widgets related to annotating the measurement result. The fifth operation may also include pressing operations with one or more fingers.

The annotation of the measurement result may include marking key positions, marking directions, marking information about major structures, etc.

The sixth operation may include operations such as clicking, swiping, long-pressing, double-clicking, etc., on applications and/or widgets related to generating the examination report. The sixth operation may also include pressing operations with one or more fingers.

The examination report may be a paper report printed for the scanned object and/or an electronic report stored in the system (e.g., the system 100, the system 1000, the system 1100).

Potential beneficial effects of the embodiments of the present disclosure include, but are not limited to: (1) Various types of collection devices are used to gather diverse user information, which ensures the comprehensiveness, accuracy, and diversity of user information collection. (2) The second fingerprint information is matched with the first fingerprint information comprehensively and accurately. After successful matching, the medical imaging device is controlled through the execution function corresponding to the second fingerprint information, which allows the user to control the medical imaging device without pressing multiple buttons or combinations of buttons, thereby enhancing the convenience and efficiency of medical imaging device control, reducing the count of buttons on the medical imaging device control panel, and lowering manufacturing costs and user operational workload. (3) Before the user uses the medical imaging device, the operator state of the medical imaging device may be obtained through the methods described in the embodiments of the present disclosure. If the operator state is not null, priority is given to matching the second fingerprint information with the personal fingerprint data, thereby reducing the computational load during the fingerprint information matching verification process and improving system calculation speed. (4) The mutually exclusive anti-interrupt mechanism is configured in the medical imaging device, which not only effectively prevents interruptions during the execution of important functions, but also prevents the execution of mutually exclusive operations. For example, the execution functions of medical imaging optimization and scanning guidance page displaying may be mutually exclusive and may not be executed simultaneously. The status function table provides a basis for determining whether to execute operations related to the function corresponding to the second fingerprint information, reducing the impact of mis-touch and mis-operation. (5) Configuring and adjusting the layout of the user interface of the medical imaging device through various interface components increases the flexibility of the layout of the user interface of the medical imaging device, enhances application scalability, reduces redundant information, simplifies operating procedures, improves operational efficiency, facilitates user operations, and enhance user efficiency. (6) Utilizing fingerprint information to differentially display the user interface meets the needs and/or preferences of different users, improving operational efficiency, facilitating user operations, and enhancing the usage efficiency of the medical imaging device.

The basic concepts have been described above, and it is apparent that to a person skilled in the art, the above detailed disclosure is intended as an example only and does not constitute a limitation of the present disclosure. Although not expressly stated herein, various modifications, improvements, and amendments may be made to the present disclosure by those skilled in the art. Such modifications, improvements, and amendments are suggested in the present disclosure, so such modifications, improvements, and amendments remain within the spirit and scope of the exemplary embodiments of the present disclosure.

Also, the present disclosure uses specific words to describe the embodiments of the present disclosure. For example, “an embodiment,” “one embodiment,” and/or “some embodiments” are meant to refer to a certain feature, structure, or characteristic associated with at least one embodiment of the present disclosure. Accordingly, it should be emphasized and noted that “an embodiment” or “one embodiment” or “an alternative embodiment” mentioned two or more times in different places in the present disclosure do not necessarily refer to the same embodiment. Furthermore, certain features, structures, or characteristics in one or more embodiments of the present disclosure may be suitably combined.

Furthermore, unless expressly stated in the claims, the order of processing elements and sequences, the use of numerical letters, or the use of other names described herein are not intended to limit the order of the processes and methods of the present disclosure. Although a number of embodiments of the present disclosure currently considered useful are discussed in the above disclosure by way of various examples, it should be understood that such details serve illustrative purposes only, and that additional claims are not limited to the disclosed embodiments; rather, the claims are intended to cover all amendments and equivalent combinations that are consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be noted that in order to simplify the presentation of the present disclosure, and thus aid in the understanding of one or more embodiments of the present disclosure, the preceding description of embodiments of the present disclosure sometimes combines multiple features into a single embodiment, accompanying drawings, or description thereof. However, this way of disclosure does not imply that the subject matter of the present disclosure requires more features than those mentioned in the claims. Rather, claimed subject matter may lie in less than all features of a single foregoing disclosed embodiment.

In some embodiments, numeric values describing the composition and quantity of attributes are used in the description. It should be understood that such numeric values used for describing embodiments may be modified with qualifying terms such as “about,” “approximately,” or “generally.” Unless otherwise stated, “about,” “approximately,” or “generally” indicates that a variation of +20% is permitted in the described numbers. Accordingly, in some embodiments, the numerical parameters used in the disclosure and claims are approximations, which may change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

Finally, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

1. A method for controlling a medical imaging device implemented on a computing device having one or more processors and one or more storage devices, comprising:
obtaining second fingerprint information of a user through a collection device;
matching the second fingerprint information with first fingerprint information to obtain a matching result; and
determining, based on a first mapping relationship and the matching result, an execution function corresponding to the second fingerprint information, and controlling the medical imaging device to perform an operation related to the execution function corresponding to the second fingerprint information.

2. The method of claim 1, wherein the first mapping relationship is obtained through a process including:
obtaining the first fingerprint information of the user; and
configuring one or more execution functions of the medical imaging device corresponding to the first fingerprint information to generate the first mapping relationship between the first fingerprint information and the one or more execution functions corresponding to the first fingerprint information.

3. The method of claim 1, wherein
the medical imaging device includes an ultrasound device; and
the execution function includes at least one of ultrasound image optimization, ultrasound measurement, interface switching, ultrasound image annotation, coded harmonic ultrasound imaging, scanning guidance page displaying, ultrasound probe switching, ultrasound clinical application, patient registration and management, ultrasound reporting, or ultrasound image reviewing.

4. The method of claim 1, wherein:
the first fingerprint information includes fingerprint information of multiple fingers of the user;
the first mapping relationship includes a corresponding relationship between a combination of fingerprint information of two or more fingers of the user and one or more execution functions corresponding to the first fingerprint information, or a corresponding relationship between a combination of a fingerprint image of one finger of the user with different pressing parameters and the one or more execution functions corresponding to the first fingerprint information,
wherein the pressing parameters include at least one of a count of presses, a pressing strength, a pressing duration, or a pressing angle of the user on the collection device.

5. The method of claim 1, wherein the matching the second fingerprint information with first fingerprint information to obtain a matching result includes:
obtaining an operator state of the medical imaging device; and
in response to determining that the operator state is not null, matching the second fingerprint information with personal fingerprint data corresponding to the operator state to obtain a first matching result, or in response to determining that the operator state is null, matching the second fingerprint information with global fingerprint data to obtain a second matching result.

6. The method of claim 5, wherein the determining, based on a first mapping relationship and the matching result, an execution function corresponding to the second fingerprint information includes:
in response to determining that the first matching result is successful,
determining first target fingerprint information in the first fingerprint information that matches the second fingerprint information,
determining, based on the first mapping relationship, a first target execution function corresponding to the first target fingerprint information, and
designating the first target execution function as the execution function corresponding to the second fingerprint information;
in response to determining that the first matching result is unsuccessful, matching the second fingerprint information with the global fingerprint data to obtain the second matching result; or
in response to determining that the second matching result is successful,
determining second target fingerprint information in the global fingerprint data that matches the second fingerprint information,
determining, based on a mapping relationship associated with the second target fingerprint information, a second target execution function corresponding to the second target fingerprint information,
designating the second target execution function as the execution function corresponding to the second fingerprint information, and
updating the operator state to be not null; or
in response to the determining that the second matching result is unsuccessful,
notifying the user that the second matching result is unsuccessful,
configuring the execution function of the medical imaging device corresponding to the second fingerprint information, and
updating the first mapping relationship to generate a second mapping relationship.

7. The method of claim 1, wherein the controlling the medical imaging device to perform an operation related to the execution function corresponding to the second fingerprint information includes:
determining, based on a mutual exclusion anti-interruption mechanism, whether to execute the operation related to the execution function corresponding to the second fingerprint information.

8. The method of claim 7, wherein the determining, based on a mutual exclusion anti-interruption mechanism, whether to execute the operation related to the execution function corresponding to the second fingerprint information includes:
determining whether the medical imaging device is in a frozen state to obtain a first judgment result; and
determining, based on the first judgment result, whether to execute the operation related to the execution function corresponding to the second fingerprint information.

9. The method of claim 1, wherein the controlling the medical imaging device to perform an operation related to the execution function corresponding to the second fingerprint information includes:
in response to determining that a pressing strength of the user on the collection device is less than a threshold, displaying the execution function corresponding to the second fingerprint information to the user; or
in response to determining that the pressing strength of the user on the collection device is greater than the threshold, executing the execution function corresponding to the second fingerprint information.

10. The method of claim 1, wherein
the first fingerprint information includes a fingerprint image and information related to the fingerprint image,
the information related to the fingerprint image includes a target object corresponding to the fingerprint image and a pressing parameter of the target object,
the target object includes at least one of a finger, a palm, a glove, or a prosthesis of the user, and
the pressing parameter includes at least one of a count of presses, a pressing strength, a pressing duration, or a pressing angle of the user on the collection device.

11. The method of claim 1, further comprising:
displaying a user interface including an interface component corresponding to the second fingerprint information, wherein
the interface component corresponds to one or more functions associated with the medical imaging device or an operation performed on the medical imaging device, and
a type of the interface component includes at least one of an application, an application widget, or a program dock.

12-14. (canceled)

15. A system for controlling an interface of a medical imaging device, comprising:
a processor, and
a display device configured to display a user interface including an interface component, wherein
the interface component corresponds to one or more functions associated with the medical imaging device or one or more operations performed on the medical imaging device;
the processor is configured to control the medical imaging device based on an operation for the interface component; and
a type of the interface component includes at least one of an application, an application widget, or a program dock.

16-19. (canceled)

20. The system of claim 15, wherein:
the application corresponds to at least one function of the one or more functions associated with the medical imaging device;
the application widget corresponds to at least one of information related to the application or at least one operation related to the application; and
the program dock includes one or more of the application and the application widget, and the program dock is displayed on an interface corresponding to the application.

21. The system of claim 20, wherein the application widget has multiple types, and one of the multiple types corresponds to a sequence of the one or more functions associated with the medical imaging device executed in order, and each function in the sequence corresponds to a function associated with the medical imaging device.

22. The system of claim 15, wherein
the interface component is configured to be adjustable based on a user operation, a user level, and/or a user permission.

23. The system of claim 22, wherein an adjustment of the interface component is performed through user initiatively or performed automatically based on user operation data.

24. The system of claim 22, further comprising a collection device configured to obtain second fingerprint information of a user, and the processor is further configured to perform at least one of the following operations:
executing a corresponding function or a corresponding operation based on the second fingerprint information;
determining the user level or the user permission based on the second fingerprint information; and
executing a corresponding function or a corresponding operation based on the user level or the user permission.

25. The system of claim 22, wherein the display device is further configured to recognize second fingerprint information of a user, and the processor is further configured to perform at least one of the following operations:
executing a corresponding function or a corresponding operation based on the second fingerprint information;
determining the user level or the user permission based on the second fingerprint information; and
executing a corresponding function or a corresponding operation based on the user level or the user permission.

26. The system of claim 24, wherein the executing a function or a corresponding operation based on the second fingerprint information includes:
matching the second fingerprint information with first fingerprint information to obtain a matching result;
determining, based on a first mapping relationship and the matching result, an execution function corresponding to the second fingerprint information, and controlling the medical imaging device to perform an operation related to the execution function.

27-29. (canceled)

30. A method for controlling an interface of a medical imaging device, comprising:
displaying a user interface including an interface component through a display device; and
receiving an operation for the interface component through one or more processors and controlling the medical imaging device, wherein
the interface component corresponds to one or more functions associated with the medical imaging device or one or more operations performed on the medical imaging device,
a type of the interface component includes at least one of an application, an application widget, or a program dock.

31-39. (canceled)
